(19) United States
(12) Patent Application Publication
(10) Pub. No.: US 2008/0287430 A1
(43) Pub. Date: Nov. 20, 2008

(54) Furan Compounds Useful As Ep1 Receptor Antagonists

(76) Inventors: Rino Antonio Bit, Essex (GB); Gerard Martin Paul Giblin, Essex (GB); Adrian Hall, Essex (GB); David Nigel Hurst, Essex (GB); Ian Reginald Kilford, Essex (GB); Tiziana Scoccitti, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/912,321

(22) PCT Filed: Apr. 24, 2006
(86) PCT No.: PCT/EP2006/003808

§ 371 Date: Jun. 26, 2008

(30) Foreign Application Priority Data
Apr. 26, 2005 (GB) 0508460.3

(51) Int. Cl. A61K 31/341; C07D 307/40; C07D 405/06; A61K 31/496; C07D 413/12; A61P 25/00; A61K 31/5377; A61K 31/4985; A61K 31/443; C07D 471/04

(52) U.S. Cl. 514/231.5; 549/484; 546/284.7; 544/379; 544/350; 514/461; 514/336; 514/254.1; 514/249; 544/152

(57) ABSTRACT

Compounds of formula (I) or a pharmaceutically acceptable derivative thereof

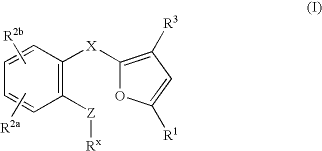

wherein X, Z, R¹, R^{2a}, R^{2b}, R³, and R^{x} are as defined in the specification, a process for the preparation of such compounds, pharmaceutical compositions comprising such compounds and the use of such compounds in medicine.

This invention relates to furan compounds, to processes for their preparation, to pharmaceutical compositions containing them and to their use in medicine, in particular their use in the treatment of conditions mediated by the action of PGE₂ at the EP₁ receptor.

Prostaglandin receptors, including the EP₁₋₄, DP, FP, IP and TP receptors are the effector proteins for the products (prostaglandins) downstream of COX-1/2 activation (PGE₂, PGD2, PGF2a, PGI2 and thromboxane respectively). The NSAIDS (non-steroidal anti-inflammatory drugs) are indiscriminate cyclooxygenase inhibitors and reduce the levels of these prostaglandins. This in turn reduces the action of the prostaglandins at their respective receptors. In view of the relatively large number of receptors affected, the pharmacology of the NSAIDS is complex.

The EP₁, receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion.

We have now found a novel group of compounds which bind with high affinity to the EP₁ receptor. These compounds are antagonists of the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S. Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. In The American Physiological Society (1994, 267, R289-R-294), studies suggest that PGE₂-induced hyperthermia in the rat is mediated predominantly through the EP₁ receptor.

The TP (also known as TxA₂) receptor is a prostanoid receptor subtype stimulated by the endogenous mediator thromboxane. Activation of this receptor results in various physiological actions primarily incurred by its platelet aggregatory and smooth muscle constricting effects, thus opposing those of prostacyclin receptor activation.

TP receptors have been identified in human kidneys (G. P. Brown et al, Prostaglandins and other lipid mediators, 1999, 57, 179-188) in the glomerulus and extraglomerular vascular tissue. Activation of TP receptors constricts glomerular capillaries and suppresses glomerular filtration rates (M. D. Breyer et al, Current Opinion in Nephrology and Hypertension, 2000, 9, 23-29), indicating that TP receptor antagonists could be useful for renal dysfunction in glomerulonephritis, diabetes mellitus and sepsis.

Activation of TP receptors induces bronchoconstriction, increase in microvascular permeability, formation of mucosal oedema and mucus secretion, typical characteristic features of bronchial asthma (T. Obata et al, Clinical Review of Allergy, 1994, 12(1), 79-93). TP antagonists have been investigated as potential asthma treatments resulting in, for example, orally active Seratrodast (AA-2414) (S. Terao et al, Yakugaku Zasshi, 1999, 119(5), 377-390). Ramatroban is another TP receptor antagonist currently undergoing phase III clinical trials as an anti-asthmatic compound.

Antagonists at the TP receptor have been shown to have a gastroprotective effect. In rats it has been shown that SQ 33961 and BM 13505 inhibit gastric lesions induced by taurocholate acid, aspirin or indomethacin (E. H. Ogletree et al, Journal of Pharmacology and Experimental Therapeutics, 1992, 263(1), 374-380.

Certain compounds of the present invention also exhibit antagonism at the TP receptor and are therefore indicated to be useful in treating conditions mediated by the action of thromboxane at the TP receptor. Such conditions include those disclosed in WO 2004/039807 (Merck Frosst Canada & Co) which is incorporated herein by reference, and include respiratory diseases e.g. asthma, allergic diseases, male erectile dysfunction, thrombosis, renal disorders and gastric lesions.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005) and WO2005/108369 (17 Nov. 2005) disclose compounds as being useful in the treatment of prostaglandin mediated diseases.

A. Hall et al, Bioorg. Med. Chem. Lett., 2006, 16, 2666-2671 discloses biaryl heterocyclic EP₁ receptor agonists.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

Accordingly the present invention provides compounds of formula (I):

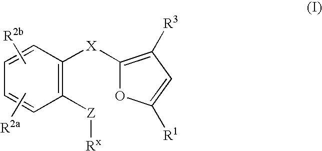

wherein:

### X is CR⁷R⁸, O, S, SO, or SO₂;

### Z is O, S, SO or SO₂;

R^{x} is C₂₋₁₀alkyl optionally substituted by C₁₋₃alkoxy, optionally substituted C₂₋₁₀alkenyl, optionally substituted C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CONR⁴R⁵, CH₂CO₂H, NHCO₂R⁶, 1,2,4-triazolyl, tetrazolyl, or CH₂tetrazolyl; or R¹ is imidazolyl, or pyrazolyl wherein optionally the imidazole or pyrazole ring is fused to give an optionally substituted bicyclic or tricyclic ring system;
R^{2a} and R^{2b} are independently selected from hydrogen, halo, CN, SO₂alkyl, SR⁴, NO₂, optionally substituted alkyl, optionally substituted alkoxy, optionally substituted aryl, and optionally substituted heteroaryl;
R³ is hydrogen or optionally substituted C₁₋₃alkyl;
R⁴ is hydrogen or optionally substituted alkyl;
R⁵ is hydrogen or optionally substituted alkyl, optionally substituted aryl, optionally substituted heteroaryl, optionally substituted SO₂aryl, optionally substituted SO₂alkyl, optionally substituted SO₂heteroaryl, optionally substituted CQ^{a}Q^{b} aryl, or optionally substituted CQ^{a}Q^{b}heteroaryl; or
R⁴ and R⁵ together with the nitrogen to which they are attached form a benzimidazolyl or 4-phenylmethylpiperazinyl group;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
and derivatives thereof;
provided that:
when R¹ is NHCO₂R⁶, R^{x} represents optionally substituted alkyl;
when R¹ is benzimidazolyl it is unsubstituted on the 1-position; and
when R¹ is benzimidazolyl, optional substituents on the 4 or 7 position are selected from halogen, CH₂OH and CO₂H.

Optional substituents for alkyl, alkenyl or alkynyl groups include OH, CO₂R^{y}, NR^{y}R^{z}, (O), OC₁₋₆alkyl or halo, wherein R^{y} and R^{z} are independently selected from hydrogen and C₁₋₆alkyl. An alkyl group may be substituted by one or more optional substituents, for example up to 5, 4, 3, or 2 optional substituents.

Optional substituents for aryl, heteroaryl or heterocyclyl moieties as a group or part of a group are selected from optionally substituted C₁₋₆alkyl, optionally substituted C₁₋₆alkoxy and halogen.

Suitably X is CR⁷R⁸ or O.

Suitably Z is O.

Suitably R⁴ is hydrogen.

Suitably R⁵ is hydrogen or optionally substituted alkyl, optionally substituted aryl, optionally substituted heteroaryl, optionally substituted SO₂aryl, optionally substituted SO₂alkyl, optionally substituted SO₂heteroaryl, optionally substituted CQ^{a}Q^{b}aryl, or optionally substituted CQ^{a}Q^{b}heteroaryl;

In one aspect R¹ represents CO₂H, CONHR⁵, CH₂CO₂H, 1,2,4-triazolyl, tetrazolyl, or CH₂tetrazolyl; or R¹ represents imidazolyl, or pyrazolyl wherein optionally the imidazole or pyrazole ring is fused to give an optionally substituted bicyclic or tricyclic ring system;

In one aspect the present invention provides compound of formula (I) wherein:

### X is CR⁷R⁸, or O;

### Z is O;

R^{x} is C₂₋₁₀alkyl, C₂₋₁₀alkenyl, C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CONHR⁵, CH₂CO₂H, NHCO₂R⁶, 1,2,4-triazolyl, tetrazolyl, or CH₂tetrazolyl; or R¹ is imidazolyl, or pyrazolyl wherein optionally the imidazole or pyrazole ring is fused to give an optionally substituted bicyclic or tricyclic ring system;
R^{2a} and R^{2b} are independently selected from hydrogen, halo, CN, SO₂alkyl, SR⁴, NO₂, optionally substituted alkyl, optionally substituted alkoxy, optionally substituted aryl, and optionally substituted heteroaryl;
R³ is hydrogen or optionally substituted C₁₋₃alkyl;
R⁵ is hydrogen or optionally substituted alkyl, optionally substituted aryl, optionally substituted heteroaryl, optionally substituted SO₂aryl, optionally substituted SO₂alkyl, optionally substituted SO₂heteroaryl, optionally substituted CQ^{a}Q^{b} aryl, or optionally substituted CQ^{a}Q^{b} heteroaryl;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine; and derivatives thereof;
provided that:
when R¹ is NHCO₂R⁶, R^{x} represents optionally substituted alkyl;
when R¹ is benzimidazolyl it is unsubstituted on the 1-position; and
when R¹ is benzimidazolyl, optional substituents on the 4 or 7 position are selected from halogen, CH₂OH and CO₂H.

In one aspect X is CR⁷R⁸.

Suitably R¹ is CO₂H, CONH₂, CONHC₁₋₆alkyl, optionally substituted CONHPh, optionally substituted CONHSO₂Ph, optionally substituted CONHCH₂phenyl, CONHCH₂pyridyl, CONHCH₂naphthyl, optionally substituted CO-piperazinyl, CO-benzimidazolyl, CH₂CO₂H, NHCO₂C₁₋₆alkyl, 1,2,4-triazolyl, imidazolyl, or pyrazolyl wherein the imidazole or pyrazole may be optionally fused to give a bicyclic or tricyclic ring system.

Optional substituents for R¹ when CONHPh include up to three substituents independently selected from F, Cl, Br, phenyl, C₁₋₄alkyl, C₁₋₄alkoxy, morpholinyl, imidazolyl, triazolyl and SO₂C₁₋₄alkyl. Optional substituents for R¹ when CONHCH₂Ph include up to three substituents independently selected from F, Cl, Br, phenyl, pyridyl, C₁₋₄alkyl, C₁₋₄alkoxy, CN, CF₃, CONH₂, N(CH₃)₂, CN or OCH₂O.

When R¹ is 1,2,4-triazolyl, imidazolyl, or pyrazolyl, in one aspect the rings are attached to the furan ring through a carbon atom.

When R¹ is imidazole or pyrazole optionally fused to give a bicyclic or tricyclic ring system, examples include benzimidazole, 1H-imidazolo[4,5-b]pyridine, 1H-imidazo[4,5-c]pyridine, 1H-imidazo[4,5-b]pyrazine, 1H-thieno[3,4-d]imidazole, 1H-imidazo[4,5-c]quinoline and 1,7-dihydroimidazo[4,5-f]indazole. These ring systems may be optionally substituted with one or two substituents.

When R¹ is substituted benzimidazole, preferably it is substituted on the 5 and/or 6 positions.

When R¹ is benzimidazole, in one aspect it is attached to the furan ring through the 2-position carbon atom.

Optional substituents for such fused ring systems include halogen, C₁₋₄alkyl, ethenyl, OCH₃, CF₃, CH₂OH, CH₂NR^{a}R^{b}, CH₂CH₂NR^{a}R^{b}, piperazinemethyl, and morpholinyl; wherein R^{a} is hydrogen, methyl or ethyl and R^{b} is optionally substituted C₁₋₆alkyl or R^{a} and R^{b} together with the nitrogen atom to which they are attached form a morpholinyl, piperidinyl, pyrrolidinyl, piperazinylmethyl, or piperazinylethyl group. Optional substitutents for R^{b} include OH, CN, OCH₃ and tetrahydropyranyl.

In one aspect, when R¹ is imidazole or pyrazole optionally fused to give a bicyclic or tricyclic ring system, optional substituents for the fused ring systems include F, Cl, Br, C₁₋₄alkyl, ethenyl, CF₃, OCH₃, CH₂OH, CH₂CH₂OH, piperazinemethyl, morpholinyl, CH₂NR^{a}R^{b}, CH₂CH₂NR^{a}R^{b}; wherein R^{a} is hydrogen, methyl or ethyl and R^{b} is C₁₋₅alkyl, e.g. methyl, ethyl, propyl, iso-propyl or neopentyl, CH₂CH₂OH, CH₂CH₂OCH₃, CH₂CH₂CN, or CH₂tetrahydropyranyl.

In one aspect R^{2a} is hydrogen.

Suitably R^{2b} is selected from halogen e.g. Cl.

Preferably R^{2b} is positioned 1,4-relative to the Z substituent and 1,3-relative to the methylene furyl moiety.

Preferably R^{2a} is hydrogen, and R^{2b} is Cl and is positioned 1,4-relative to the Z substituent and 1,3-relative to the methylene furyl moiety.

In a particular aspect Z is O; R^{2a} is hydrogen; R^{2b} is Cl and is positioned 1,4-relative to the Z substituent; and 1,3-relative to the furan moiety; and is CR⁷R⁸.

Suitably R³ is hydrogen or methyl.

When R^{x} represents optionally substituted C₂₋₁₀alkyl the group may be, for example, propyl, 1-ethylpropyl, 1-methylethyl, 2-methylpropyl, 3-methylbutyl, 2-ethylbutyl, cyclopentyl, cyclopropylmethylene, cyclopentylmethylene, and cyclohexylmethylene. In one aspect the alkyl group is C₃₋₈alkyl and is unsubstituted.

When R^{x} represents optionally substituted CQ^{a}Q^{b}-heterocyclyl, optionally substituted CQ^{a}Q^{b}-bicyclic heterocyclyl or optionally substituted CQ^{a}Q^{b}-aryl, suitably R^{x} includes optionally substituted CH₂-heterocyclyl, e.g. CH₂-isoxazolyl and CH₂-pyridyl, optionally substituted CH₂-bicyclic heterocyclyl or optionally substituted CH₂-aryl e.g. optionally substituted CH₂-phenyl. Optional substituents for CH₂-phenyl include one, two or three substituents each independently selected from Cl and F. Optional substituents for CH₂-heterocyclyl include Cl and CH₃.

In one aspect R^{x} represents optionally substituted C₂₋₈alkyl, optionally substituted CH₂-heterocyclyl or optionally substituted CH₂-phenyl.

Suitably R⁵ includes hydrogen, C₁₋₆alkyl, optionally substituted naphthyl, optionally substituted phenyl, SO₂Ph, optionally substituted CH₂Ph, and optionally substituted CH₂pyridyl.

When R⁵ is optionally substituted phenyl it may be substituted by up to three substituents. Examples of optional substituents when R⁶ is phenyl include F, Cl, Br, SO₂C₁₋₄alkyl, heterocyclyl e.g. morpholine, triazole, imidazole, C₁₋₄alkyl, and phenyl.

When R⁵ is optionally substituted CH₂Ph, the phenyl ring may be substituted with up to three substituents selected from, for example, F, Cl, Br, CF₃, CN, C₁₋₄alkoxy, methylenedioxy, pyridyl, amido, and di(C₁₋₄alkyl)amino.

Suitably R⁶ includes C₁₋₄alkyl, e.g. 1,1-dimethylethyl.

Suitably R⁷ includes hydrogen.

Suitably R⁸ includes hydrogen.

Suitably Q^{a} is hydrogen.

Suitably Q^{b} is hydrogen.

Compounds of formula (I) include the compounds of examples 1 to 169 and derivatives thereof.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine. The term “alkyl” as a group or part of a group means a straight, branched or cyclic alkyl group or combinations thereof. Unless hereinbefore defined, examples of alkyl include C₁₋₈alkyl, for example methyl, ethyl, n-propyl, iso-propyl, n-butyl, sec-butyl, iso-butyl, t-butyl, pentyl, hexyl, 1,1-dimethylethyl, cyclopropyl, cyclopentyl or cyclohexyl or combinations thereof such as cyclopropylmethylene, cyclohexylmethylene and cyclopentylmethylene.

When used herein the term “cycloalkyl” means a cyclic alkyl group comprising up to eight carbon atoms in a ring.

The term “alkenyl” means linear or branched structures and combinations thereof, of the indicated number of carbon atoms, having at least one carbon-to-carbon double bond, wherein hydrogen may be replaced by an additional carbon to carbon double bond. C₃₋₈alkenyl, for example, includes 2-methyl-2-propenyl and the like.

The term “alkynyl” means linear or branched structures and combinations thereof, of the indicated number of carbon atoms, having at least one carbon-to-carbon triple bond. C₃₋₈alkynyl, for example, includes propynyl and the like.

The term “alkoxy” as a group or as part of a group means a straight, branched or cyclic chain alkoxy group. Unless hereinbefore defined “alkoxy” includes C₁₋₈alkoxy, e.g. methoxy, ethoxy, n-propoxy, iso-propoxy, n-butoxy, sec-butoxy, iso-butoxy, t-butoxy, pentoxy, hexyloxy, cyclopentoxy or cyclohexyloxy. In one aspect “alkoxy” is C₁₋₆ alkoxy.

The term “heterocyclyl” as a group or as part of a group means an aromatic or non-aromatic five or six membered ring which contains from 1 to 4 heteroatoms selected from nitrogen, oxygen or sulfur and unsubstituted or substituted by, for example, up to three substituents, preferably one or two substituents. Examples of 5-membered heterocycles include furan, tetrahydrofuran, thiophene, tetrahydrothiophene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, and tetrazole. Examples of 6-membered heterocycles include pyran, tetrahydropyran, pyridine, piperidine, dioxane, morpholine, dithiane, thiomorpholine, pyridazine, pyrimidine, pyrazine, piperazine, and triazine.

The term “heterocyclyloxy” as a group or as part of a group refers to an “—O-heterocyclyl” group, wherein the term “heterocyclyl” is as defined above.

The term “aliphatic heterocyclyl” as a group or as part of a group means an aliphatic five or six membered ring which contains 1 or 2 heteroatoms selected from nitrogen, oxygen or sulfur and is unsubstituted or substituted by, for example, up to three substituents, preferably one or two substituents.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

The term “aryloxy” as a group or as part of a group refers to an “—O-aryl” group, wherein the term “aryl” is as defined above.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate be substituted by one or two substituents selected from hydrogen and C₁₋₈alkyl, preferably hydrogen and C₁₋₆alkyl, more preferably hydrogen.

Compounds of formula (I) can be prepared as set forth in the following schemes and in the Examples. The following processes form another aspect of the present invention.

Compounds of formula (I) wherein R¹ is CO₂H, hereinafter referred to as compounds of Formula (Ia), may be prepared by the general route below:

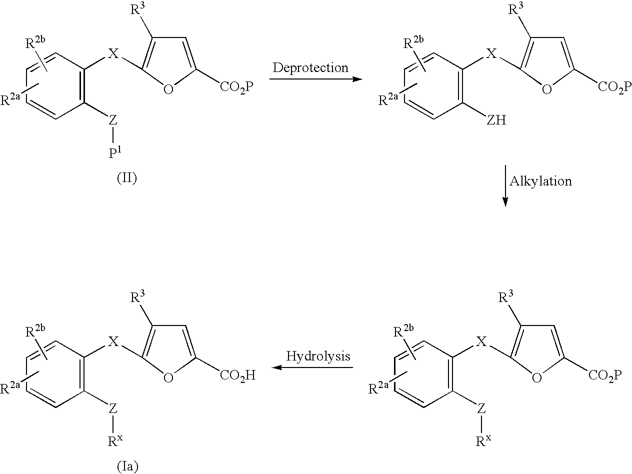

wherein X, Z, R^{2a}, R^{2b}, R³, and R^{x} are as defined for compounds of formula (I);
and P and P¹ are protecting groups.

Compounds of formula (Ia) may be prepared from an intermediate of formula (II) by removal of P¹ followed by reaction with a suitable source of R^{x} wherein R^{x} is as defined for a compound of formula (I). Suitable sources of R^{x} include R^{x}Br. Suitable reaction conditions when the source of R^{x} is R^{x}Br include heating in the presence of a base e.g. potassium carbonate in a suitable solvent e.g. acetone or N,N-dimethylformamide, followed by removal of protecting group P.

Suitable protecting groups will be known to the skilled person. Suitably P is C₁₋₄alkyl or optionally substituted benzyl. Suitable protecting groups when Z is O include C₁₋₄alkyl or benzyl.

Suitable deprotection methods will be known to the skilled person. Conditions for the deprotection of an ester to give the corresponding carboxylic acid are known to those skilled in the art and include heating in the presence of a suitable base, e.g. aqueous sodium hydroxide, in a solvent e.g. an alcohol.

Removal of the protecting group P¹ can be achieved for example through treatment with boron tribromide in a suitable solvent, for example dichloromethane at reduced temperature.

It will be recognised to those skilled in the art that the compounds of formula (I) wherein R¹ is other than CO₂H can be derived from the carboxylic acid (Ia). Compounds wherein R¹ is CONR⁴R⁵, such as amides or acylsulfonamides, can be prepared by activation of the carboxylic acid, for example by forming the acid chloride (for example by reaction of the carboxylic acid with thionyl chloride) followed by reaction with an amine or a sulfonamide respectively. Other derivatives may be accessed by using the Curtius reaction (P. A. S. Smith, Org. React. 3, 337-449 (1946) and J. H. Saunders, R. J. Slocombe, Chem. Rev. 43, 205 (1948)), followed by deprotection of the resulting carbamate and reaction with a carboxylic acid derivative such as an acid chloride. Compounds where a methylene group has been inserted between the furan ring and the carboxylic acid group may be prepared using the Arndt-Eistert reaction (F. Arndt, B. Eistert, Ber. 68, 200 (1935); W. E. Bachmann, W. S. Struve, Org. React. 1, 38-62 (1942), G. B. Gill, Comp. Org. Syn. 3, 888-889 (1991), T. Aoyama, Tetrahedron Letters, 21, 4461 (1980)) and the methods described in the examples. It will be recognised to those skilled in the art that a carboxylic acid group may be converted to a pyrazole, triazole or imidazole group by a sequence of well known functional group transformations such as those described in the Examples. Tetrazoles may be formed from carboxylic acids by converting the carboxylic acid to the primary amide, for example by reaction with sulfonyl chloride followed by ammonia, followed by dehydration of the amide to the nitrile, for example by heating in phosphorous oxychloride, followed by reaction with azide.

Compounds of formula (I) wherein R¹ is an imidazole moiety fused to give an optionally substituted bicyclic or tricyclic ring system [hereinafter referred to as compounds of formula (Ib)] may be prepared from compounds of formula (III) in accordance with the following scheme:

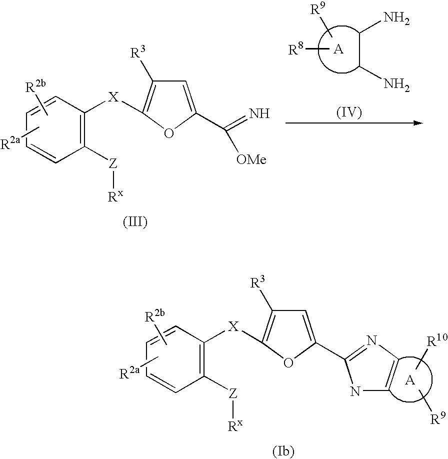

wherein X, Z, R^{2a}, R^{2b}, R³, and R^{x} are as defined for compounds of formula (I); A represents e.g. phenyl, pyridine, quinoline, or thiophene, and R⁹ and R¹⁰ each represent hydrogen or a substituent.

Suitable reaction conditions for the preparation of a compound of formula (Ib) include heating the intermediates together in a suitable solvent e.g. ethanol.

Compounds of formula (II) when X is CH₂ may be prepared by known methods via Friedel-Crafts alkylation of a suitable phenyl derivative with a chloromethylfuran intermediate as described in the Examples and by the following scheme:

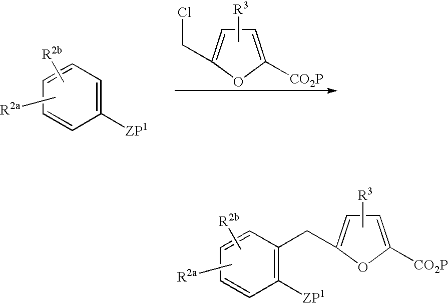

wherein Z, R^{2a}, R^{2b}, R³, P and P¹ are as defined for compounds of formula (I); and P and P¹ are protecting groups as defined above.

Suitable reaction conditions include carrying out the reaction in a suitable solvent such as nitromethane in the presence of aluminium chloride. Suitable chloromethylfuran intermediates are commercially available, or may be readily prepared by known techniques from commercially available starting materials such as the corresponding hydroxymethyl derivative. Suitable phenyl derivatives are commercially available or may be readily prepared from commercially available starting materials by known functional group transformations.

When X═O, compounds of formula (II) may be prepared by reacting a nitrofuran intermediate with the required phenoxide in accordance with the scheme below:

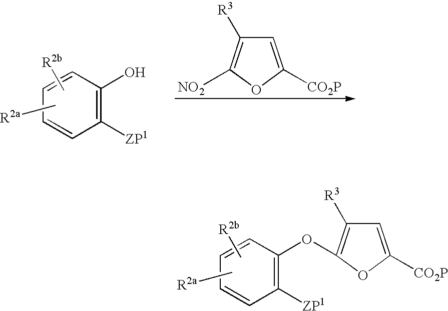

Z, R^{2a}, R^{2b}, R³, P and P¹ are as defined for compounds of formula (I); and P and P¹ are protecting groups as defined above.

Suitable reaction conditions for the preparation of compounds of formula (II) wherein X═O include heating the desired nitrofuroate together with the appropriate phenoxide in a suitable solvent such as N,N-dimethylformamide.

Compounds of formula (III) may be prepared from the corresponding carboxylic acid of formula (Ia) by known methods, for example as described in the examples. Suitable methods include the conversion of a compound of formula (Ia) to the corresponding acid chloride using for example thionyl chloride, followed by conversion to the amide using ammonia. The amide may be converted to the corresponding nitrile using phosphorus oxychloride. Treatment with sodium methoxide in methanol provides the compound of formula (III).

Diamines of formula (IV) are commercially available, or may be prepared by known methods from commercially available starting materials.

Compounds of formula (Ib) wherein R¹ is a benzimidazole may also be prepared from the reaction of a diamine of formula (IV) with a compound of formula (I) wherein R¹ is CHO.

Compounds of formula (Ib) wherein R¹ is benzimidazole may be functionalised on the benzimidazole ring using methods described in the examples and in the scheme below:

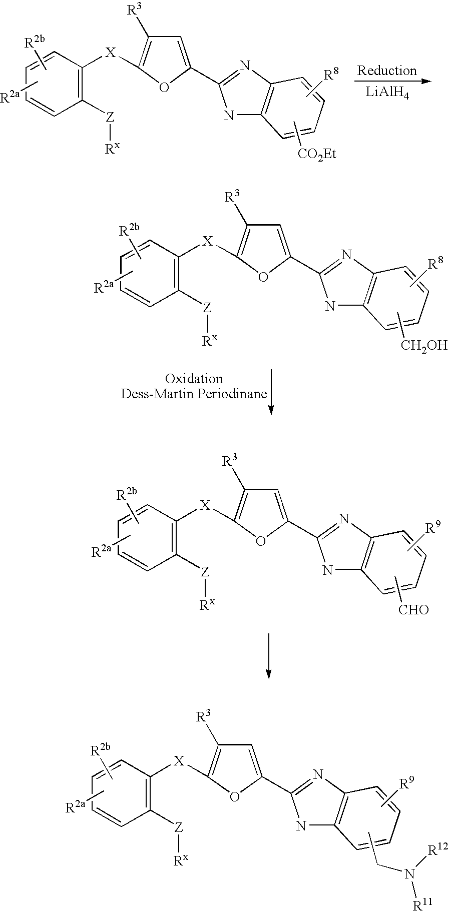

wherein X, Z, R^{2a}, R^{2b}, R³, and R^{x} are as hereinbefore defined for compounds of formula (I). R⁹ is hydrogen or a substituent and R¹¹ and R¹² are independently selected from hydrogen, and optionally substituted C₁₋₄alkyl, or R¹¹ and R¹² together with the nitrogen atom to which they are attached form a heterocyclyl ring optionally containing another heteroatom selected from O, NH, NC₁₋₄alkyl, or S.

Accordingly the present invention also provides a process for the preparation of a compound of formula (I) or a derivative thereof:

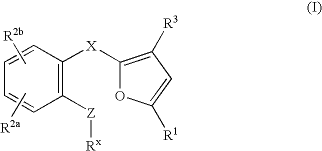

wherein:

### X is CR⁷R⁸, O, S, SO, or SO₂;

### Z is O, S, SO or SO₂;

R^{x} is C₂₋₁₀alkyl optionally substituted by C₁₋₃alkoxy, optionally substituted C₂₋₁₀alkenyl, optionally substituted C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CONR⁴R⁵, CH₂CO₂H, NHCO₂R⁶, 1,2,4-triazolyl, tetrazolyl, or CH₂tetrazolyl; or R¹ is imidazolyl, or pyrazolyl wherein optionally the imidazole or pyrazole ring is fused to give an optionally substituted bicyclic or tricyclic ring system;
R^{2a} and R^{2b} are independently selected from hydrogen, halo, CN, SO₂alkyl, SR⁴, NO₂, optionally substituted alkyl, optionally substituted alkoxy, optionally substituted aryl, and optionally substituted heteroaryl;
R³ is hydrogen or optionally substituted C₁₋₃alkyl;
R⁴ is hydrogen or optionally substituted alkyl;
R⁵ is hydrogen or optionally substituted alkyl, optionally substituted aryl, optionally substituted heteroaryl, optionally substituted SO₂aryl, optionally substituted SO₂alkyl, optionally substituted SO₂heteroaryl, optionally substituted CQ^{a}Q^{b} aryl, or optionally substituted CQ^{a}Q^{b}heteroaryl; or
R⁴ and R⁵ together with the nitrogen to which they are attached form a benzimidazolyl or 4-phenylmethylpiperazinyl group;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
provided that:
when R¹ is NHCO₂R⁶, R^{x} represents optionally substituted alkyl;
when R¹ is benzimidazolyl it is unsubstituted on the 1-position; and
when R¹ is benzimidazolyl, optional substituents on the 4 or 7 position are selected from halogen, CH₂OH and CO₂H.
comprising:
alkylating a compound:

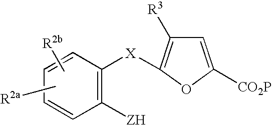

with a source of R^{x};
wherein R^{2a}, R^{2b}, R³, R^{x}, Z, X are as defined above for a compound of formula (I) and P is a protecting group; and;
in any order
effecting deprotection;
if necessary, converting the group CO₂H or CO₂P to another group R¹; and/or
forming a derivative thereof.

Suitable sources of R^{x} include R^{x}Br wherein R^{x} is as defined for compounds of formula (I). These compounds are commercially available, or may be readily prepared by known transformations of commercially available compounds.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opiods (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendonitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendonitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; monoaminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

In addition to activity at the EP₁ receptor, certain compounds of the present invention and pharmaceutically acceptable derivatives thereof exhibit antagonism of the TP receptor and are therefore indicated to be useful in treating conditions mediated by the action of thromboxane at the TP receptor. Conditions mediated by the action of thromboxane at the TP receptor include renal disorders, asthma, or gastric lesions.

In certain situations it is envisaged that the administration of a compound exhibiting antagonism of TP receptors in combination with a compound exhibiting antagonism of EP₁ receptors may be advantageous.

Certain compounds of the invention are selective for EP₁ over EP₃.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

It will be appreciated to those skilled in the art that where compounds are named as hydrochloride salts the stoichiometry of the isolated reaction products is undetermined due to the nature of their preparation. Compounds have therefore been named as hydrochlorides and denoted as xHCl, where x is 0-3 and represents the stoichiometry of said salt.

### Abbreviations

AcOH, acetic acid, Bn (benzyl), Bu, Pr, Me, Et (butyl, propyl, methyl, ethyl), DMSO (dimethyl sulfoxide), DCM/MDC (dichloromethane), DME (ethylene glycol dimethyl ether), DMF (N,N-dimethylformamide), EDTA (ethylenediaminetetraacetic acid), EtOAc (ethyl acetate), EtOH (ethanol), HPLC (High pressure liquid chromatography), IPA (isopropanol), LCMS (Liquid chromatography/Mass spectroscopy), MDAP (Mass Directed Auto Preparation), MeOH (methanol), ML (mother liquor), NMR (Nuclear Magnetic Resonance (spectrum)), NMP (n-methylpyrrolidone), Ph (phenyl), pTSA (para-toluene sulphonic acid), RT/Rt (retention time), SM (starting material), SPE (Solid Phase Extraction—silica cartridge chromatography), TBAF (tetrabutylammonium fluoride), TBME (tertiary butyl methyl ether), THF (tetrahydrofuran), s, d, dd, t, q, m, br (singlet, doublet, double doublet, triplet, quartet, multiplet, broad.)

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

Flash Master II is an automated chromatography system using commercial prepacked columns. Biotage is a chromatography system using commercial pre-packed silica gel cartridges. The term FLEX (Parallel Flex) when used herein refers to a parallel HPLC purification system.

### LCMS

The following conditions were used for LCMS in the preparation of the examples.
Column: 3.3 cm×4.6 mm ID, 3 μm ABZ+PLUS
Flow Rate: 3 ml/min
Injection Volume: 5 μl
Temp: Room temperature
UV Detection Range: 215 to 330 nm

### Solvents:

A: 0.1% Formic Acid+10 mMolar Ammonium Acetate.
B: 95% Acetonitrile+0.05% Formic Acid

| | | | |
|---|---|---|---|
| Gradient: | Time | A % | B % |

| | | |
|---|---|---|
| 0.00 | 100 | 0 |
| 0.70 | 100 | 0 |
| 4.20 | 0 | 100 |
| 5.30 | 0 | 100 |
| 5.50 | 100 | 0 |

All retention times are measured in minutes.

### Preparation of Intermediates

### Ethyl 5-(5-chloro-2-methoxybenzyl)-2-furoate

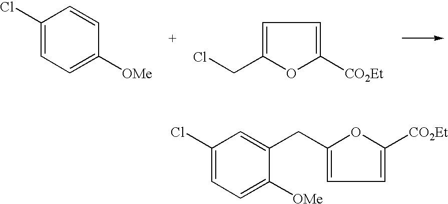

Aluminium chloride (3.2 g, 24 mmol) was added over 2 minutes to a solution of 4-chloroanisole (3.422 g, 24 mmol) and ethyl 5-chloromethyl-2-furancarboxylate (3.772 g, 20 mmol) in nitromethane (75 ml) at room temperature and stirred for 24 hours. The resulting mixture was carefully diluted with water/diethyl ether and the organic phase dried (magnesium sulphate) and purified by chromatography on silica gel eluting with ethyl acetate/hexane (7:93) to give the title compound as a light coloured oil (4.85 g).

¹H NMR (CDCl₃) δ: 1.36 (t, 3H), 3.80 (s, 3H), 4.00 (s, 2H), 4.34 (q, 2H), 6.05 (d, 1H), 6.79 (d, 1H), 7.08-7.10 (m, 2H), 7.19 (dd, 1H).

### Ethyl 5-[(5-chloro-2-hydroxyphenyl)methyl]-2-furancarboxylate

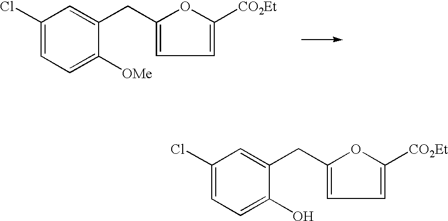

Boron tribromide (8.17 g, 32.6 mmol) was added to a solution of ethyl 5-(5-chloro-2-methoxybenzyl)-2-furoate (4.8 g, 16.3 mmol) in dichloromethane (10 ml) at −78° C. and allowed to warm to room temperature. After 30 minutes the mixture was poured onto ice and diluted with ethyl acetate/water. The organic phase was washed with saturated sodium bicarbonate, dried (magnesium sulphate), evaporated and crystallised from diethyl ether/hexane to give the title compound as a white solid (3.51 g).

LC/MS: Rt=2.99, [MH−]279.1, 281.1

### Ethyl 5-[(5-chloro-2-{[(2,4-difluorophenyl)methyl]oxy}phenyl)methyl]-2-furancarboxylate

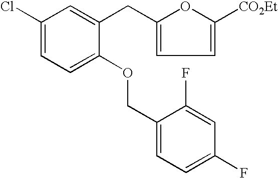

A mixture of ethyl 5-[(5-chloro-2-hydroxyphenyl)methyl]-2-furancarboxylate (900 mg, 3.2 mmol), 2,4-difluorobenzyl bromide (800 mg, 3.85 mmol) and potassium carbonate (690 mg, 5 mmol) in acetone (10 ml) was refluxed for 2 hours. The reaction mixture was cooled, filtered and the solvent evaporated from the filtrate. Purification of the residue by flash chromatography eluting with 10% ethyl acetate in hexane gave the title compound as a colourless oil (1.1 g). LCMS Rt=3.78 min [MH⁺] 407.

The following compounds were prepared in a similar manner to ethyl 5-[(5-chloro-2-{[(2,4-difluorophenyl)methyl]oxy}phenyl)methyl]-2-furancarboxylate using the appropriate starting materials.

| | Name |
|---|---|
| 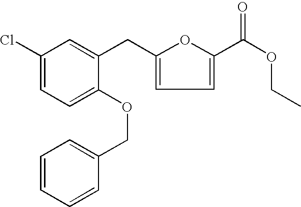 | Ethyl 5-({5-chloro-2-[(phenylmethyl)oxy]phenyl}methyl)-2-furancarboxylate |
| 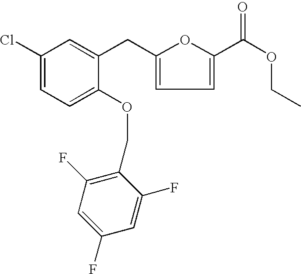 | Ethyl 5-[(5-chloro-2-{[(2,4,6-trifluorophenyl)methyl]oxy}phenyl)methyl]-2-furancarboxylate |
| 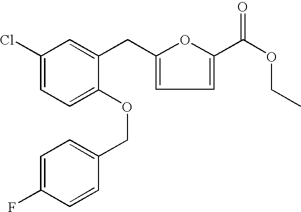 | Ethyl 5-[(5-chloro-2-{[(4-fluorophenyl)methyl]oxy}phenyl)methyl]-2-furancarboxylate |
| 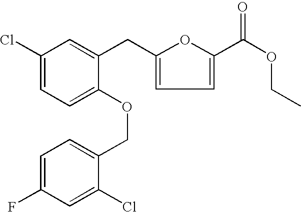 | Ethyl 5-[(5-chloro-2-{[(2-chloro-4-fluorophenyl)methyl]oxy}phenyl)methyl]-2-furancarboxylate |
| 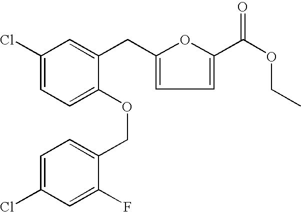 | Ethyl 5-[(5-chloro-2-{[(4-chloro-2-fluorophenyl)methyl]oxy}phenyl)methyl]-2-furancarboxylate |
| 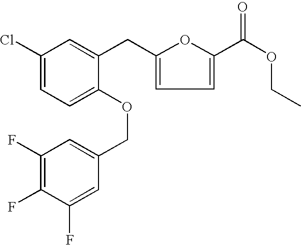 | Ethyl 5-[(5-chloro-2-{[(3,4,5-trifluorophenyl)methyl]oxy}phenyl)methyl]-2-furancarboxylate |
| 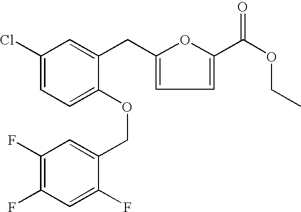 | Ethyl 5-[(5-chloro-2-{[(2,4,5-trifluorophenyl)methyl]oxy}phenyl)methyl]-2-furancarboxylate |
| 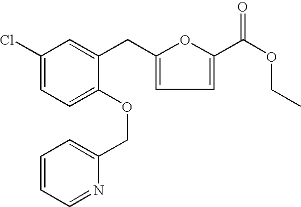 | Ethyl 5-({5-chloro-2-[(2-pyridinylmethyl)oxy]phenyl}methyl)-2-furancarboxylate |
| 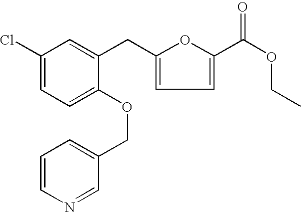 | Ethyl 5-({5-chloro-2-[(3-pyridinylmethyl)oxy]phenyl}methyl)-2-furancarboxylate |
| 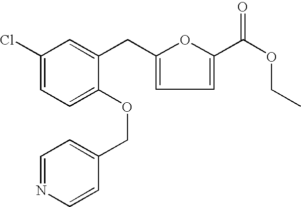 | Ethyl 5-({5-chloro-2-[(4-pyridinylmethyl)oxy]phenyl}methyl)-2-furancarboxylate |

### Ethyl 5-({5-chloro-2-[(2-methylpropyl)oxy]phenyl}methyl)-2-furancarboxylate

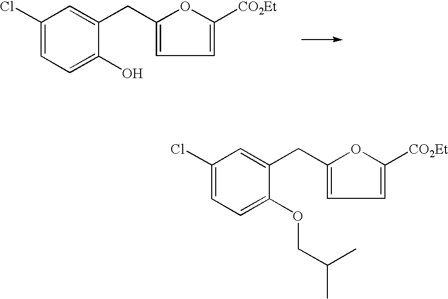

A mixture of ethyl 5-[(5-chloro-2-hydroxyphenyl)methyl]-2-furancarboxylate (3.48 g, 12.4 mmol), isobutyl bromide (3.43 g, 25 mmol) and potassium carbonate (5.52 g, 40 mmol) in dimethylformamide (40 ml) was stirred and heated at 80° C. for 6 hours when isobutyl bromide (0.5 g, 3.65 mmol) was added and heating continued for a further hour. The resulting mixture was cooled, diluted with diethyl ether/water and the organic phase washed three times with water, dried (magnesium sulphate), evaporated and purified by chromatography on silica gel eluting with ethyl acetate/hexane (1:19) to give the title compound as a colourless oil (3.81 g).

LC/MS: Rt=3.66, [MH⁺] 337.2

The following compounds were prepared by alkylation of ethyl 5-[(5-chloro-2-hydroxyphenyl)methyl]-2-furancarboxylate using the same procedure as above for the preparation of ethyl 5-({5-chloro-2-[(2-methylpropyl)oxy]phenyl}methyl)-2-furancarboxylate.

| | Name | LC/MS |
|---|---|---|
| 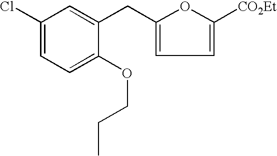 | Ethyl 5-{[5-chloro-2-(propyloxy)phenyl]methyl}-2-furancarboxylate | Rt = 3.51[MH]⁺ 323.2 |
| 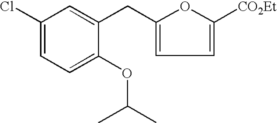 | Ethyl 5-({5-chloro-2-[(1-methylethyl)oxy]phenyl}methyl)-2-furancarboxylate | Rt = 3.45[MH]⁺ 323.2 |
| 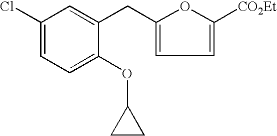 | Ethyl 5-{[5-chloro-2-(cyclopropyloxy)phenyl]methyl}-2-furancarboxylate | Rt = 3.45[MH]⁺ 335.1 |
| 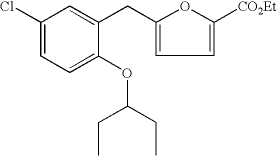 | Ethyl 5-({5-chloro-2-[(1-ethylpropyl)oxy]phenyl}methyl)-2-furancarboxylate | Rt = 3.70[MH]⁺ 351.2 |
| 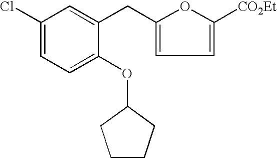 | Ethyl 5-{[5-chloro-2-(cyclopentyloxy)phenyl]methyl}-2-furancarboxylate | Rt = 3.65[MH]⁺ 349.2 |
| 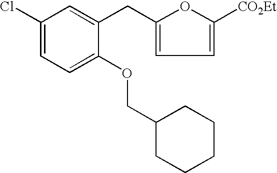 | Ethyl 5-({5-chloro-2-[(cyclohexylmethyl)oxy]phenyl}methyl)-2-furancarboxylate | Rt = 3.90[MH]⁺ 377.2 |
| 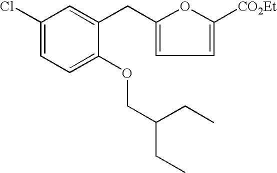 | Ethyl 5-({5-chloro-2-[(2-ethylbutyl)oxy]phenyl}methyl)-2-furancarboxylate | Rt = 3.83[MH]⁺ 366.1 |
| 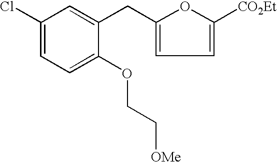 | Ethyl 5-[(5-chloro-2-{[2-(methyloxy)ethyl]oxy}phenyl)methyl]-2-furancarboxylate | Rt = 3.27[MH]⁺ 339.0 |
| 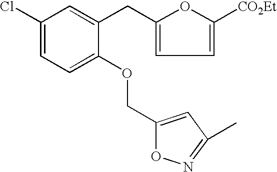 | Ethyl 5-[(5-chloro-2-{[(3-methyl-5-isoxazolyl)methyl]oxy}-phenyl)methyl]-2-furancarboxylate | Rt = 3.33[MH]⁺ 376.0 |
| 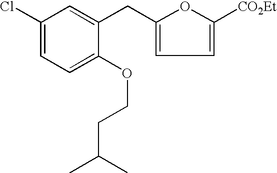 | Ethyl 5-({5-chloro-2-[(3-methylbutyl)oxy]phenyl}methyl)-2-furancarboxylate | Rt = 3.81[MH]⁺ 351.1 |
| 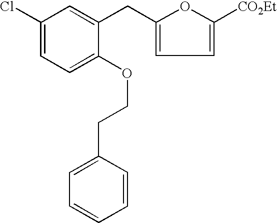 | Ethyl 5-({5-chloro-2-[(2-phenylethyl)oxy]phenyl}methyl)-2-furancarboxylate | Rt = 3.69[MH]⁺ 385.1 |
| 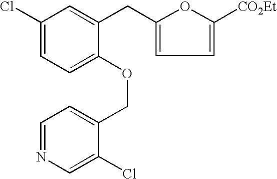 | Ethyl 5-[(5-chloro-2-{[(3-chloro-4-pyridinyl)methyl]oxy}phenyl)methyl]-2-furancarboxylate | Rt = 3.46[MH]⁺ 406.1 |

### Methyl 5-{[5-chloro-2-(methyloxy)phenyl]oxy}-2-furancarboxylate

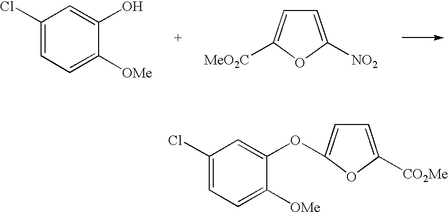

60% Sodium hydride (200 mg, 5 mmol) was added to a stirred solution of 5-chloro-2-methoxyphenol in dimethylformamide (8 ml) and the mixture stirred for 10 minutes. Methyl 5-nitro-2-furoate (856 mg, 5 mmol) was added and the mixture heated at 120° C. for 90 minutes then cooled to room temperature and diluted with diethyl ether/water. The organic phase was washed with 2M sodium hydroxide and water (x2), dried (magnesium sulphate) and evaporated to give the title compound as a pale coloured oil (1.19 g).

LC/MS: Rt=3.10, [MH]⁺ 283.1

### Methyl 5-[(5-chloro-2-hydroxyphenyl)oxy]-2-furancarboxylate

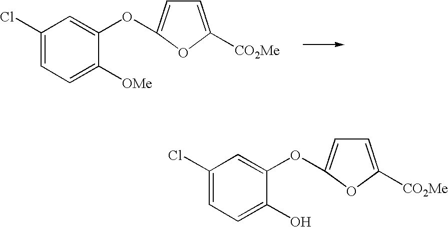

Boron tribromide (2.65 g, 10.56 mmol) was added to a solution of methyl 5-{[5-chloro-2-(methyloxy)phenyl]oxy}-2-furancarboxylate (1.18 g, 4.17 mmol) in dichloromethane (6 ml) and left at room temperature for 1 hour before being poured onto ice/ether. The organic phase was washed with saturated sodium bicarbonate solution, dried (magnesium sulphate), evaporated and purified by chromatography on silica gel eluting with ethyl acetate/hexane (1:4). The product was triturated with ether/hexane to give the title compound as a white solid (910 mg).

LC/MS: Rt=2.83, [MH]⁺ 269.1, 271.1

### Methyl 5-({5-chloro-2-[(phenylmethyl)oxy]phenyl}oxy)-2-furancarboxylate

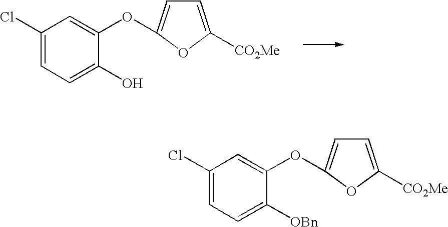

A mixture of methyl 5-[(5-chloro-2-hydroxyphenyl)oxy]-2-furancarboxylate (134 mg, 0.5 mmol), benzyl bromide (103 mg, 0.6 mmol) and potassium carbonate (138 mg, 1 mmol) in acetone (5 ml) was stirred and refluxed for 3 hours. The resulting mixture was cooled, filtered, evaporated and purified by chromatography on silica gel eluting with ethyl acetate/hexane (1:8) to give the title compound as a white solid (130 mg).

LC/MS: Rt=3.49, [MH]⁺ 359.2

### Ethyl 5-(hydroxymethyl)-3-methyl-2-furancarboxylate

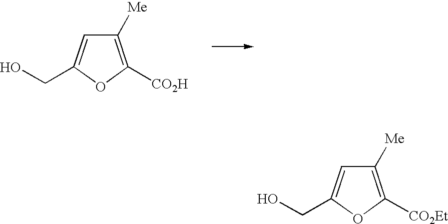

A mixture of 5-(hydroxymethyl)-3-methyl-2-furancarboxylic acid (10 g, 64.06 mmol) potassium carbonate (27.6 g, 200 mmol) and iodoethane (15.6 g, 100 mmol) in DMF (70 ml) was stirred and heated at 70° C. for 2 hours. After cooling the mixture was diluted with water/ether and the organic phase washed three times with water, dried (magnesium sulphate), evaporated and purified by chromatography on silica gel eluting with ethyl acetate/hexane (3:7) to give the title compound as a yellow/brown oil (6.8 g).

¹H NMR (CDCl₃) δ: 1.36 (t, 3H), 2.08 (s, 3H), 2.16 (br. s, 1H), 4.34 (q, 2H), 4.63 (d, 2H), 6.99 (s, 1H).

### Ethyl 5-(chloromethyl)-3-methyl-2-furancarboxylate

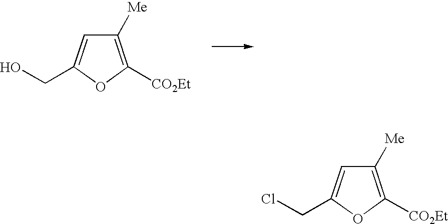

Benzotriazole (8.93 g, 75 mmol) was dissolved in thionyl chloride (8.95 g, 75 mmol) and the volume made up to 50 ml with dichloromethane. 31 ml (46.5 mmol) of this solution were added over 5 minutes to a solution of ethyl 5-(hydroxymethyl)-3-methyl-2-furancarboxylate (6.8 g, 36.96 mmol) in dichloromethane (150 ml), stirred for 15 minutes and filtered. The filtrate was washed with water and 0.5M sodium hydroxide, dried (magnesium sulphate), evaporated and purified by chromatography on silica gel eluting with ethyl acetate/hexane (6:94) to give the title compound as a colourless oil (6.74 g).

¹H NMR (CDCl₃) δ: 1.37 (t, 3H), 2.10 (s, 3H), 4.36 (q, 2H), 4.60 (s, 2H), 7.00 (s, 1H).

### Ethyl 5-{[5-chloro-2-(methyloxy)phenyl]methyl}-3-methyl-2-furancarboxylate

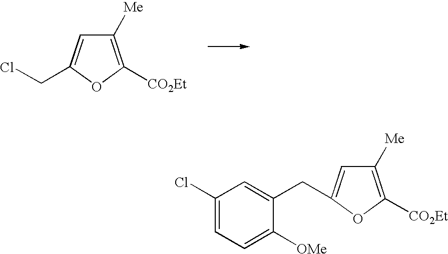

Prepared by the same method as ethyl 5-(5-chloro-2-methoxybenzyl)-2-furoate but using ethyl 5-(chloromethyl)-3-methyl-2-furancarboxylate instead of ethyl 5-(chloromethyl)-3-methyl-2-furancarboxylate.

LC/MS: Rt=3.47, [MH]⁺ 309.1

### Ethyl 5-[(5-chloro-2-hydroxyphenyl)methyl]-3-methyl-2-furancarboxylate

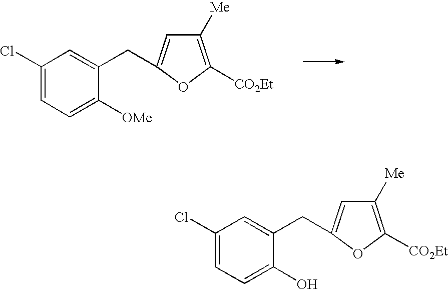

Prepared by the same method as ethyl 5-[(5-chloro-2-hydroxyphenyl)methyl]-2-furancarboxylate but using ethyl 5-{[5-chloro-2-(methyloxy)phenyl]methyl}-3-methyl-2-furancarboxylate instead of ethyl 5-(5-chloro-2-methoxybenzyl)-2-furoate.

LC/MS: Rt=3.11, [MH]⁺ 295.0

### Ethyl 5-({5-chloro-2-[(2-methylpropyl)oxy]phenyl}methyl)-3-methyl-2-furancarboxylate

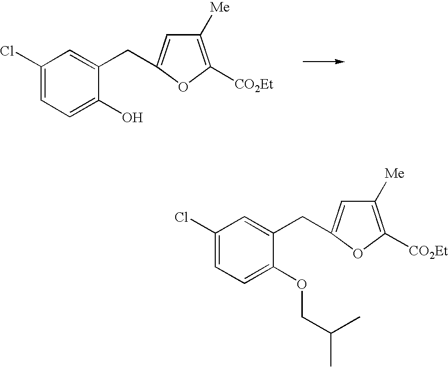

Prepared by the same method as ethyl 5-({5-chloro-2-[(2-methylpropyl)oxy]phenyl}methyl)-2-furancarboxylate but using ethyl 5-[(5-chloro-2-hydroxyphenyl)methyl]-3-methyl-2-furancarboxylate instead of ethyl 5-[(5-chloro-2-hydroxyphenyl)methyl]-2-furancarboxylate.

LC/MS: Rt=3.81, [MH]⁺ 351.1

### Ethyl 5-({5-chloro-2-[(phenylmethyl)oxy]phenyl}methyl)-3-methyl-2-furancarboxylate

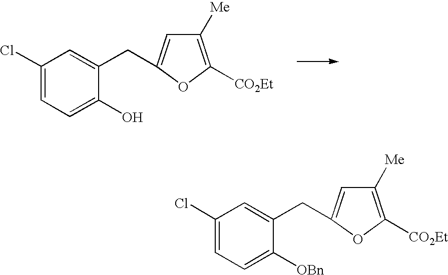

A mixture of ethyl 5-[(5-chloro-2-hydroxyphenyl)methyl]-3-methyl-2-furancarboxylate (550 mg, 1.87 mmol) benzyl bromide (359 mg, 2.1 mmol) and potassium carbonate (414 mg, 3 mmol) in acetone (10 ml) was stirred and refluxed for 3 hours. After cooling the mixture was filtered, evaporated, triturated with cold hexane and filtered to give the product as a white solid (660 mg). LC/MS: Rt=3.70, [MH]⁺ 385.1

### 5-({5-Chloro-2-[(phenylmethyl)oxy]phenyl}methyl)-N-methyl-N-(methyloxy)-2-furancarboxamide

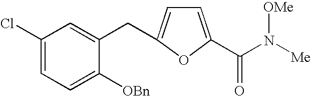

A solution of 5-({5-chloro-2-[(phenylmethyl)oxy]phenyl}methyl)-2-furancarboxylic acid (685 mg, 2 mmol), hydroxybenzotriazole (337 mg, 2.2 mmol), N,O-dimethylhydroxylamine hydrochloride (390 mg, 4 mmol), triethylamine (505 mg, 5 mmol) and N-(3-dimethylaminopropyl)-N′-ethylcarbodiimide hydrochloride (479 mg, 2.5 mmol) in dichloromethane (10 ml) was stirred at room temperature for 5 hours. The resulting mixture was washed with 2M hydrochloric acid and sodium bicarbonate solution and the organic phase dried (magnesium sulphate) and evaporated to give the title compound as a colourless gum (740 mg). LC/MS: Rt=3.39, [MH]⁺ 386.4, 388.3.

### 1-[5-({5-Chloro-2-[(phenylmethyl)oxy]phenyl}methyl)-2-furanyl]ethanone

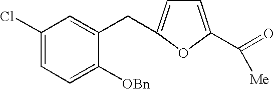

3M Methylmagnesium bromide in ether (0.8 ml, 2.4 mmol) was added to a solution of 5-({5-chloro-2-[(phenylmethyl)oxy]phenyl}methyl)-N-methyl-N-(methyloxy)-2-furancarboxamide (693 mg, 1.8 mmol) in THF (5 ml) and left at room temperature. The resulting mixture was carefully diluted with 2M hydrochloric acid/ether and the organic phase dried (magnesium sulphate), evaporated and purified by chromatography on silica gel eluting with ethyl acetate/hexane (8:92) to give the title compound as a colourless gum (451 mg).

LC/MS: Rt=3.45, [MH]⁺ 341.2, 343.2.

### Methyl [5-({5-chloro-2-[(Phenylmethyl)oxy]phenyl}methyl)-2-furanyl]acetate

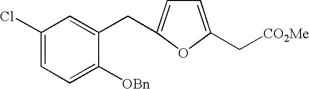

60% Perchloric acid (0.076 ml) was added to a stirred solution of 1-[5-({5-chloro-2-[(phenylmethyl)oxy]phenyl}methyl)-2-furanyl]ethanone (560 mg, 1.64 mmol) and thallium (III) nitrate trihydrate (802 mg, 1.81 mmol) in methanol (7.5 ml) and stirred for 20 hours. The resulting suspension was filtered and the filtrate diluted with diethyl ether/water. The organic phase was washed with saturated sodium bicarbonate solution, dried (magnesium sulphate), evaporated and purified by chromatography on silica gel eluting with ethyl acetate/hexane (1:8) to give the title compound as a yellow gum (231 mg).

LC/MS: Rt=3.58, [MH]⁺ 371.2.

### Standard Hydrolysis Procedure

The ester was dissolved in ethanol (5 ml) and 2M sodium hydroxide (2 ml) and left at room temperature for 4 hours. The resulting solution was evaporated to dryness, dissolved in ethyl acetate/brine and the organic phase dried (sodium sulphate), evaporated and triturated with diethyl ether/hexane to give the sodium salt as a white solid.

The following compounds were prepared using the standard hydrolysis procedure.

| Example | Structure | Name | Data |
|---|---|---|---|
| 1 | 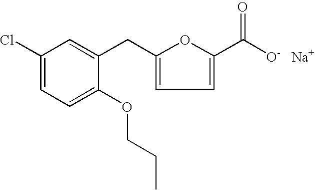 | Sodium 5-{[5-chloro-2-(propyloxy)phenyl]methyl}-2-furancarboxylate | LC/MS Rt = 3.09[MH]⁻ 293.2 |
| 2 | 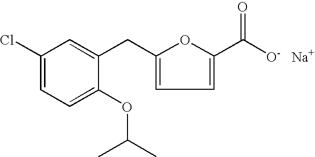 | Sodium 5-({5-chloro-2-[(1-methylethyl)oxy]phenyl}methyl)-2-furancarboxylate | LC/MS Rt = 3.05[MH]⁻ 293.2 |
| 3 | 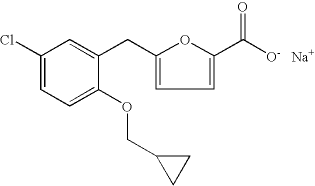 | Sodium 5-({5-chloro-2-[(cyclopropylmethyl)oxy]phenyl}methyl)-2-furancarboxylate | LC/MS Rt = 3.08[MH]⁻ 305.2 |
| 4 | 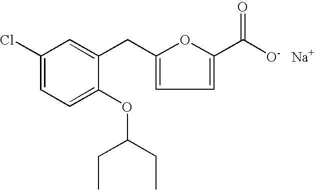 | Sodium 5-({5-chloro-2-[(1-ethylpropyl)oxy]phenyl}methyl)-2-furancarboxylate | LC/MS Rt = 3.29[MH]⁻ 321.2 |
| 5 | 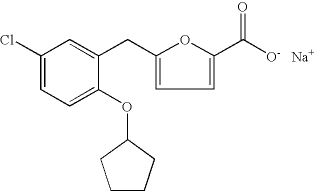 | Sodium 5-{[5-chloro-2-(cyclopentyloxy)phenyl]methyl}-2-furancarboxylate | LC/MS Rt = 3.23[MH]⁻ 319.2 |
| 6 | 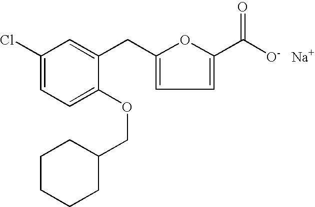 | Sodium 5-({5-chloro-2-[(cyclohexylmethyl)oxy]phenyl}methyl)-2-furancarboxylate | LC/MS Rt = 3.48[MH]⁻ 347.2 |
| 7 | 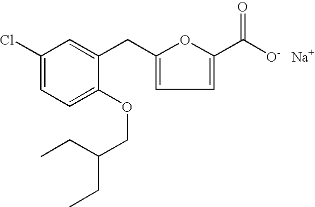 | Sodium 5-({5-chloro-2-[(2-ethylbutyl)oxy]phenyl}methyl)-2-furancarboxylate | LC/MS Rt = 3.42[MH]⁻ 335.1 |
| 8 | 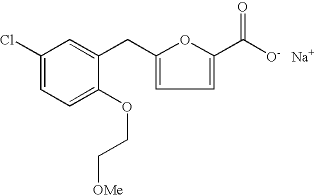 | Sodium 5-[(5-chloro-2-{[2-(methyloxy)ethyl]oxy}phenyl)methyl]-2-furancarboxylate | LC/MS Rt = 2.75[MH]⁺ 311.1 |
| 9 | 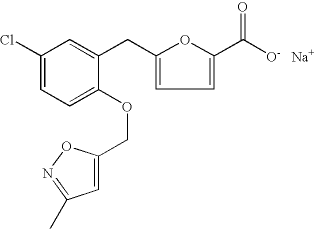 | Sodium 5-[(5-chloro-2-{[(3-methyl-5-isoxazolyl)methyl]oxy}phenyl)methyl]-2-furancarboxylate | LC/MS Rt = 2.85[MH]⁺ 348.1 |
| 10 | 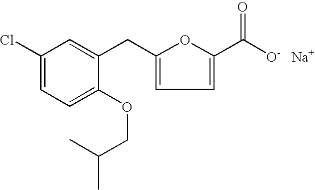 | Sodium 5-({5-chloro-2-[(2-methylpropyl)oxy]phenyl}methyl)-2-furancarboxylate | LC/MS Rt = 3.27[MH]⁺ 307.1,309.1 |
| 11 | 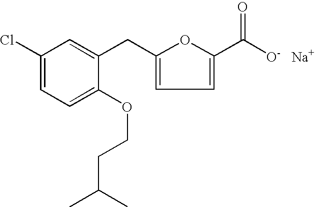 | Sodium 5-({5-chloro-2-[(3-methylbutyl)oxy]phenyl}methyl)-2-furancarboxylate | LC/MS Rt = 3.38[MH]⁺ 321.1,323.1 |
| 12 | 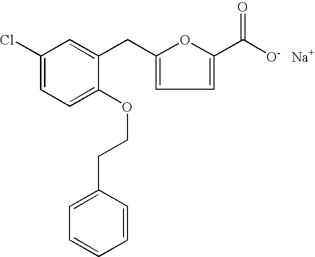 | Sodium 5-({5-chloro-2-[(2-phenylethyl)oxy]phenyl}methyl)-2-furancarboxylate | LC/MS Rt = 3.27[MH]⁺ 355.1,357.1 |
| 13 | 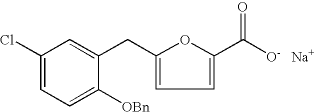 | Sodium 5-({5-chloro-2-[(phenylmethyl)oxy]phenyl}oxy)-2-furancarboxylate | LC/MS Rt = 3.09[MH]⁺ 367.0 |
| 14 | 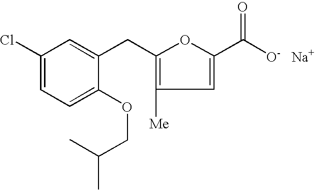 | Sodium 5-({5-chloro-2-[(2-methylpropyl)oxy]phenyl}methyl)-4-methyl-2-furancarboxylate | LC/MS Rt = 3.36[MH]⁺ 321.1,323.1 |
| 15 | 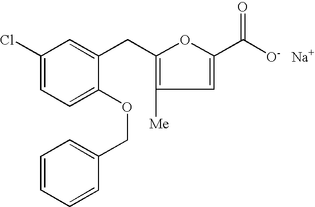 | Sodium 5-({5-chloro-2-[(phenylmethyl)oxy]phenyl}methyl)-4-methyl-2-furancarboxylate | LC/MS Rt = 3.28[MH]⁺ 357.0 |
| 16 | 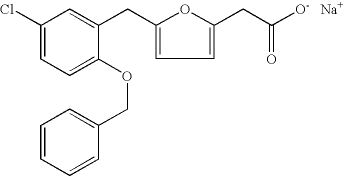 | Sodium [5-({5-chloro-2-[(phenylmethyl)oxy]phenyl}methyl)-2-furanyl]acetate | LC/MS Rt = 3.29[MH]⁺ 357.2,359.2 |
| 17 | 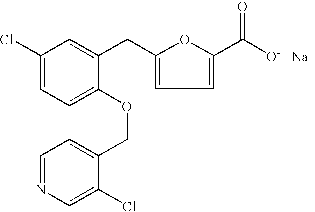 | Sodium 5-[(5-chloro-2-{[(3-chloro-4-pyridinyl)methyl]oxy}phenyl)methyl]-2-furancarboxylate | LC/MS Rt = 2.96[MH]⁻ 378.0 |
| 18 | 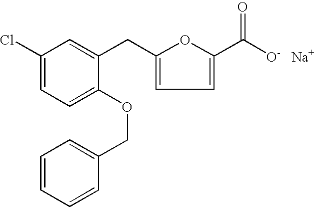 | Sodium 5-({5-chloro-2-[(phenylmethyl)oxy]phenyl}methyl)-2-furancarboxylate | ¹H NMRDMSOδ: 3.81 (2 H, s),5.10 (2 H, s),5.95 (1 H, s), 7.13-7.38(9 H, m). |
| 19 | 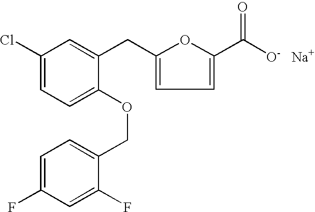 | Sodium 5-[(5-chloro-2-{[(2,4-difluorophenyl)methyl]oxy}phenyl)methyl]-2-furancarboxylate | LC/MS Rt = 3.10[MH]⁻ 377.0 |
| 20 | 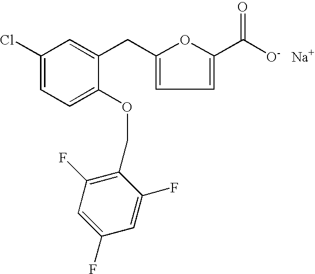 | Sodium 5-[(5-chloro-2-{[(2,4,6-trifluorophenyl)methyl]oxy}phenyl)methyl]-2-furancarboxylate | LC/MS Rt = 3.01[MH]⁻ 395.0 |
| 21 | 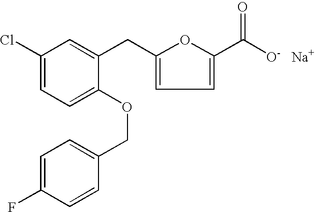 | Sodium 5-[(5-chloro-2-{[(4-fluorophenyl)methyl]oxy}phenyl)methyl]-2-furancarboxylate | LC/MS Rt = 2.94[MH]⁻ 359.0 |
| 22 | 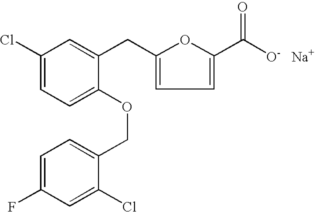 | Sodium 5-[(5-chloro-2-{[(2-chloro-4-fluorophenyl)methyl]oxy}phenyl)methyl]-2-furancarboxylate | LC/MS Rt = 2.94[MH]⁻ 393.0 |
| 23 | 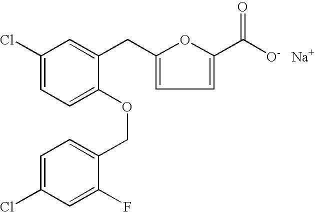 | Sodium 5-[(5-chloro-2-{[(4-chloro-2-fluorophenyl)methyl]oxy}phenyl)methyl]-2-furancarboxylate | LC/MS Rt = 3.26[MH]⁻ 393.0 |
| 24 | 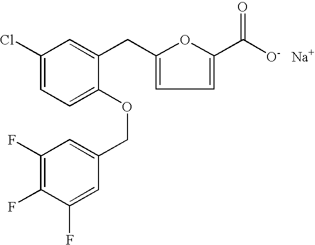 | Sodium 5-[(5-chloro-2-{[(3,4,5-trifluorophenyl)methyl]oxy}phenyl)methyl]-2-furancarboxylate | LC/MS Rt = 3.26[MH]⁻ 395.0 |
| 25 | 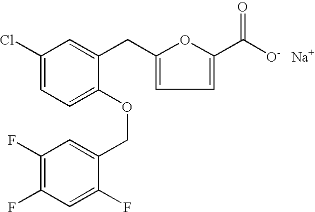 | Sodium 5-[(5-chloro-2-{[(2,4,5-trifluorophenyl)methyl]oxy}phenyl)methyl]-2-furancarboxylate | LC/MS Rt = 3.20[MH]⁻ 395.0 |
| 26 | 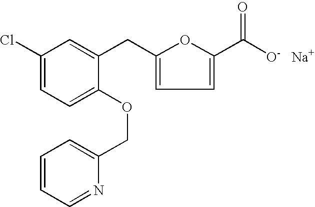 | Sodium 5-({5-chloro-2-[(2-pyridinylmethyl)oxy]phenyl}methyl)-2-furancarboxylate | LC/MS Rt = 3.20[MH]⁺ 344.0 |
| 27 | 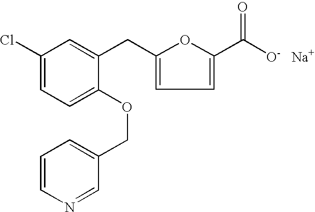 | Sodium 5-({5-chloro-2-[(3-pyridinylmethyl)oxy]phenyl}methyl)-2-furancarboxylate | LC/MS Rt = 2.05[MH]⁺ 344.0 |
| 28 | 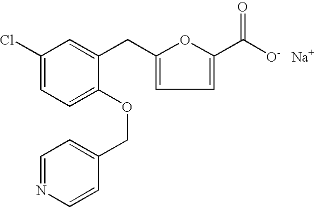 | Sodium 5-({5-chloro-2-[(4-pyridinylmethyl)oxy]phenyl}methyl)-2-furancarboxylate | LC/MS Rt = 2.05[MH]⁺ 344.0 |

### Example 29

### 1,1-Dimethylethyl[5-({5-chloro-2-[(2-methylpropyl)oxy]phenyl}methyl)-2-furanyl]carbamate

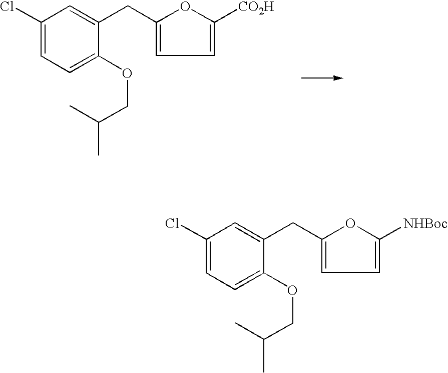

A mixture of 5-({5-chloro-2-[(2-methylpropyl)oxy]phenyl}methyl)-2-furancarboxylic acid (617 mg, 2 mmol), diphenylphosphoryl azide (550 mg, 2 mmol) and triethylamine (202 mg, 2 mmol) in t-butanol (10 ml) was stirred and heated at 85° C. for 18 hours then cooled and evaporated. The residue was dissolved in diethyl ether/water and the organic phase dried (magnesium sulphate), evaporated and purified by chromatography on silica gel eluting with ethyl acetate/hexane (1:24) then triturated with hexane to give the title compound as a white solid (210 mg). LC/MS: Rt=3.79, [MH]⁻ 378.4.

### Example 30

### 1,1-Dimethylethyl[5({5-chloro-2-[(2-methylpropyl)oxy]phenyl}methyl)-4-methyl-2-furanyl]carbamate

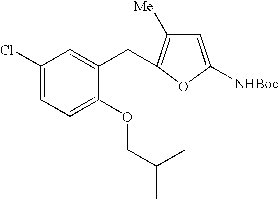

Prepared as for 1,1-dimethylethyl[5-({5-chloro-2-[(2-methylpropyl)oxy]phenyl}methyl)-2-furanyl]carbamate but using 5-({5-chloro-2-[(2-methylpropyl)oxy]phenyl}methyl)-4-methyl-2-furancarboxylic acid instead of 5-({5-chloro-2-[(2-methylpropyl)oxy]phenyl}methyl)-2-furancarboxylic acid.

### Example 31

### 5-({5-Chloro-2-[(phenylmethyl)oxy]phenyl}methyl)-4-methyl-N-(phenylmethyl)-2-furancarboxamide

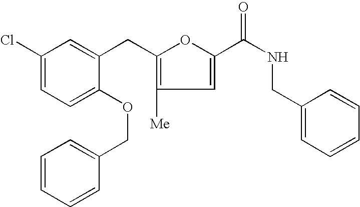

A solution of 5-({5-chloro-2-[(phenylmethyl)oxy]phenyl}methyl)-4-methyl-2-furancarboxylic acid (126 mg, 0.33 mmol), hydroxybenzotriazole (51 mg, 0.4 mmol), benzylamine (54 mg, 0.5 mmol) and N-(3-dimethylaminopropyl)-N′-ethylcarbodiimide hydrochloride (96 mg, 0.5 mmol) in dichloromethane (3 ml) was stirred at room temperature for 4 hours. The resulting mixture was diluted with ether, washed with 2M hydrochloric acid and sodium bicarbonate solution and the organic phase dried (magnesium sulphate), evaporated and triturated with hexane to give the title compound as a white solid (96 mg).

LC/MS: Rt=3.57, [MH]⁺ 446.0

The following examples were prepared in a similar manner to 5-({5-chloro-2-[(phenylmethyl)oxy]phenyl}methyl)-4-methyl-N-(phenylmethyl)-2-furancarboxamide from the appropriate intermediates using either dichloromethane or dimethylformamide as solvent.

| Example | Structure | Name | Data |
|---|---|---|---|
| 32 | 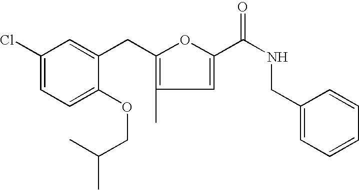 | 5-({5-Chloro-2-[(2-methylpropyl)oxy]phenyl}methyl)-4-methyl-N-(phenylmethyl)-2-furancarboxamide | LC/MSRt = 3.66[MH]⁺ 412.1,414.1 |
| 33 | 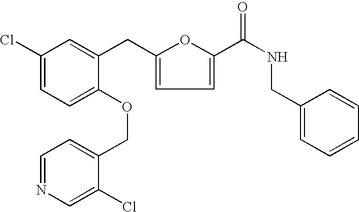 | 2-[5-({5-Chloro-2-[(phenylmethyl)oxy]phenyl}methyl)-4-methyl-2-furanyl]-N-(phenylmethyl)-acetamide | LC/MSRt = 3.29[MH]⁺ 467.2,470.2 |
| 34 | 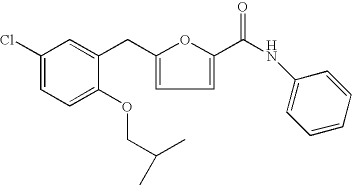 | 5-({5-Chloro-2-[(2-methylpropyl)oxy]phenyl}methyl)-N-phenyl-2-furancarboxamide | LC/MSRt = 3.64[MH]+ 384.2,386.1 |
| 35 | 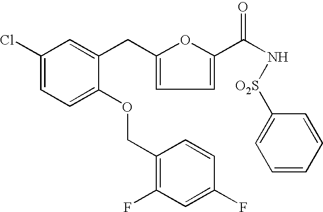 | 5-[(5-Chloro-2-{[(2,4-difluorophenyl)methyl]oxy}phenyl)methyl]-N-(phenylsulfonyl)-2-furancarboxamide | LC/MSRt = 3.18[MH]+ 518. |
| 36 | 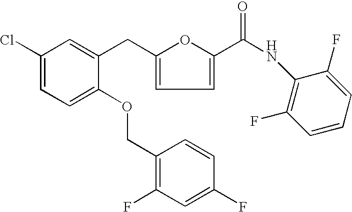 | 5-[(5-Chloro-2-{[(2,4-difluorophenyl)methyl]oxy}phenyl)methyl]-N-(2,6-difluorophenyl)-2-furancarboxamide | LC/MS:Rt = 3.53, [MH]⁺490. |
| 37 | 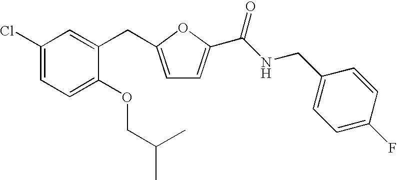 | 5-({5-Chloro-2-[(2-methylpropyl)oxy]phenyl}methyl)-N-[(4-fluorophenyl){NL methyl]-2-furancarboxamide | LC/MS Rt =3.56 min,[MH⁺] 416,418. |
| 38 | 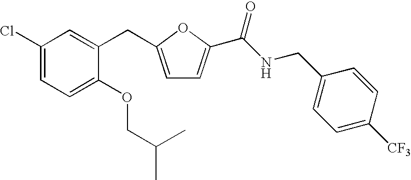 | 5-({5-Chloro-2-[(2-methylpropyl)oxy]phenyl}methyl)-N-{[4-(trifluoromethyl)phenyl]methyl}-2-furancarboxamide | LC/MS Rt =3.66 min,[MH⁺] 466,468. |
| 39 | 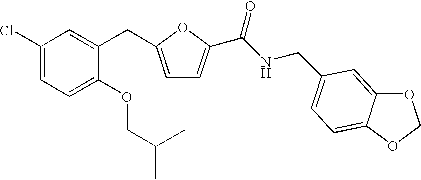 | N-(1,3-Benzodioxol-5-ylmethyl)-5-({5-chloro-2-[(2-methylpropyl)oxy]phenyl}methyl)-2-furancarboxamide | LC/MS Rt =3.50 min,[MH⁺] 442,444. |
| 40 | 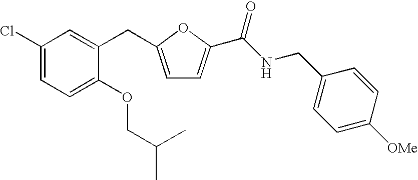 | 5-({5-Chloro-2-[(2-methylpropyl)oxy]phenyl}methyl)-N-{[4-(methyloxy)phenyl]methyl}-2-furancarboxamide | LC/MS Rt =3.53 min,[MH⁺] 428,430. |
| 41 | 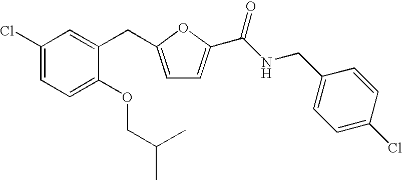 | 5-({5-Chloro-2-[(2-methylpropyl)oxy]phenyl}methyl)-N-[(4-chlorophenyl)methyl]-2-furancarboxamide | ¹H NMR(CDCl₃) δ: 0.96(6 H, d), 2.03(1 H, m), 3.68(2 H, d), 3.95(2 H, s), 4.56(2 H, d), 6.10(1 H, d), 6.54(1 H, bt) 6.75(1 H, d), 7.06(2 H, m), 7.16(1 H, dd), 7.27-7.33(4 H, m). |
| 42 | 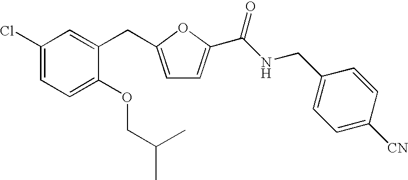 | 5-({5-Chloro-2-[(2-methylpropyl)oxy]phenyl}methyl)-N-[(4-cyanophenyl)methyl]-2-furancarboxamide | LC/MS Rt =3.45 min,[MH⁺] 423,425. |
| 43 | 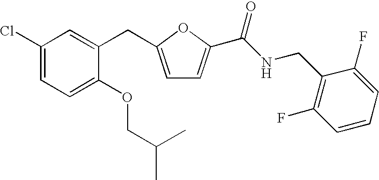 | 5-({5-Chloro-2-[(2-methylpropyl)oxy]phenyl}methyl)-N-[(2,6-difluorophenyl)methyl]-2-furancarboxamide | LC/MS Rt =3.57 min,[MH⁺] 434,436. |
| 44 | 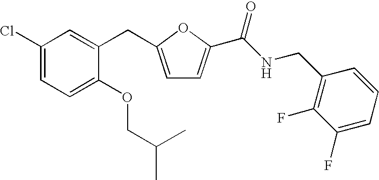 | 5-({5-Chloro-2-[(2-methylpropyl)oxy]phenyl}methyl)-N-[(2,3-difluorophenyl)methyl]-2-furancarboxamide | LC/MS Rt =3.59 min,[MH⁺] 434,436. |
| 45 | 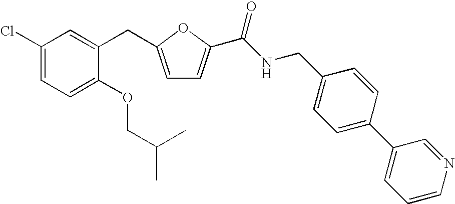 | 5-({5-Chloro-2-[(2-methylpropyl)oxy]phenyl}methyl)-N-{[4-(3-pyridinyl)phenyl]methyl}-2-furancarboxamide | LC/MS Rt =3.24 min,[MH⁺] 475,477. |
| 46 | 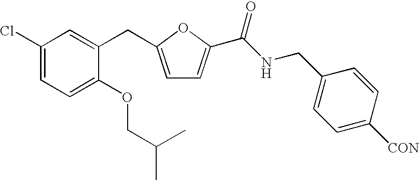 | N-{[4-(Aminocarbonyl)-phenyl]methyl}-5-({5-chloro-2-[(2-methylpropyl)oxy]phenyl}methyl)-2-furancarboxamide | LC/MS Rt =3.04 min,[MH⁺] 441,443. |
| 47 | 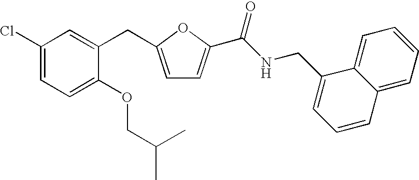 | 5-({5-Chloro-2-[(2-methylpropyl)oxy]phenyl}methyl)-N-(1-naphthalenylmethyl)-2-furancarboxamide | LC/MS Rt =3.73 min,[MH⁺] 448,450. |
| 48 | 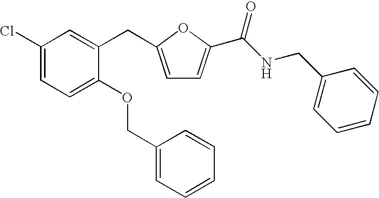 | 5-({5-Chloro-2-[(phenylmethyl)oxy]phenyl}methyl)-N-(phenylmethyl)-2-furancarboxamide | LC/MS Rt =3.46 min,[MH⁺] 430,432. |
| 49 | 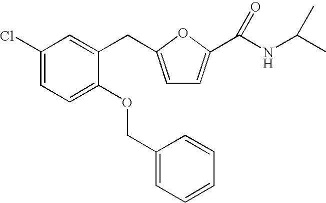 | 5-({5-Chloro-2-[(phenylmethyl)oxy]phenyl}methyl)-N-(1-methylethyl)-2-furancarboxamide | LC/MS Rt =3.35 min,[MH⁺] 384,386. |
| 50 | 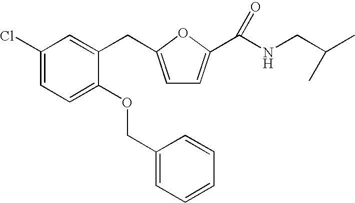 | 5-({5-Chloro-2-[(phenylmethyl)oxy]phenyl}methyl)-N-(2-methylpropyl)-2-furancarboxamide | LC/MS Rt =3.46 min,[MH⁺] 398,400. |
| 51 | 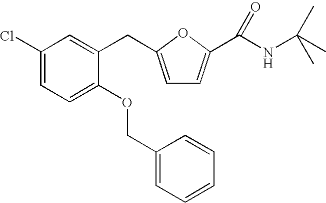 | 5-({5-Chloro-2-[(phenylmethyl)oxy]phenyl}methyl)-N-(1,1-dimethylethyl)-2-furancarboxamide | LC/MS Rt =3.57 min,[MH⁺] 398,400. |
| 52 | 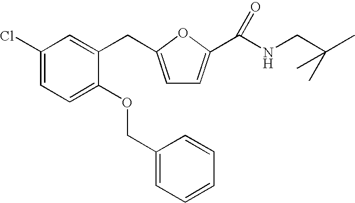 | 5-({5-Chloro-2-[(phenylmethyl)oxy]phenyl}methyl)-N-(2,2-dimethylpropyl)-2-furancarboxamide | LC/MS Rt =3.56 min,[MH⁺] 412,414. |
| 53 | 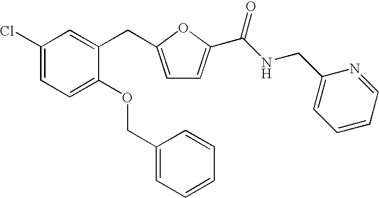 | 5-({5-Chloro-2-[(phenylmethyl)oxy]phenyl}methyl)-N-(2-pyridinylmethyl)-2-furancarboxamide | LC/MS Rt =3.01 min,[MH⁺] 433,435. |
| 54 | 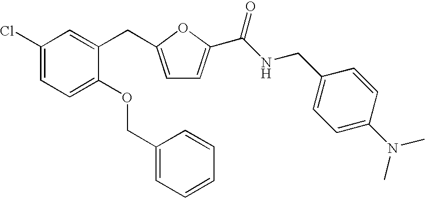 | 5-({5-Chloro-2-[(phenylmethyl)oxy]phenyl}methyl)-N-{[4-(dimethylamino)phenyl]methyl}-2-furancarboxamide | LC/MS Rt =3.26 min,[MH⁺] 475,477. |

### Example 55

### 5-({5-Chloro-2-[(2-methylpropyl)oxy]phenyl}methyl)-N-(1-methylethyl)-2-furancarboxamide

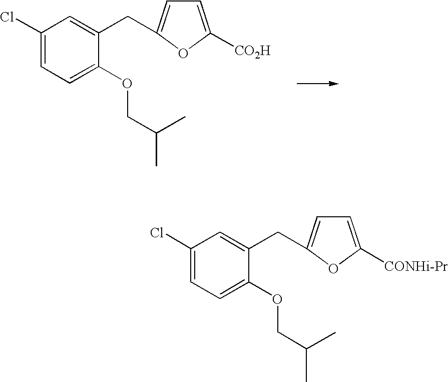

Oxalyl chloride (291 mg, 2.29 mmol) was added to a solution of 5-({5-chloro-2-[(2-methylpropyl)oxy]phenyl}methyl)-2-furancarboxylic acid (103 mg, 0.31 mmol) in dichloromethane (2 ml) and dimethylformamide (1 drop) and left at room temperature for 1 hour. The solution was evaporated to dryness, dissolved in dichloromethane (3 ml) and triethylamine (61 mg, 0.6 ml) added followed by isopropylamine (24 mg, 0.4 mmol). After 30 minutes at room temperature the mixture was diluted with diethyl ether/2M hydrochloric acid and the organic phase dried (magnesium sulphate), evaporated and the residue purified by chromatography on silica gel eluting with ethyl acetate/hexane (1:4). Trituration of the product with hexane gave the title compound as a white solid (64 mg).

LC/MS: Rt=3.44, [MH]⁺ 350.1, 352.1.

The following examples were prepared in a similar manner to 5-({5-chloro-2-[(2-methylpropyl)oxy]phenyl}methyl)-N-(1-methylethyl)-2-furancarboxamide from the appropriate intermediates.

| Examples | Structure | Name | Data |
|---|---|---|---|
| 56 | 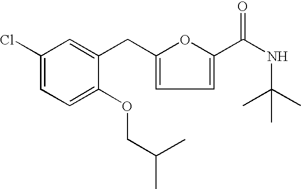 | 5-({5-Chloro-2-[(2-methylpropyl)oxy]-phenyl}methyl)-N-(1,1-dimethylethyl)-2-furancarboxamide | LC/MSRt = 3.61[MH]⁺ 364.1 |
| 57 | 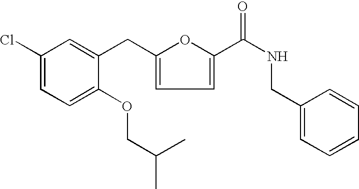 | 5-({5-Chloro-2-[(2-methylpropyl)oxy]-phenyl}methyl)-N-(phenylmethyl)-2-furancarboxamide | LC/MSRt = 3.51[MH]⁺ 398.1 |
| 58 | 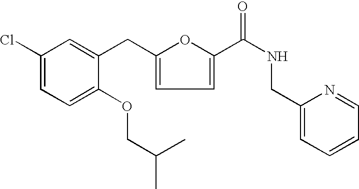 | 5-({5-Chloro-2-[(2-methylpropyl)oxy]-phenyl}methyl)-N-(2-pyridinylmethyl)-2-furancarboxamide | LC/MSRt = 2.73[MH]⁺ 399.1 |
| 59 | 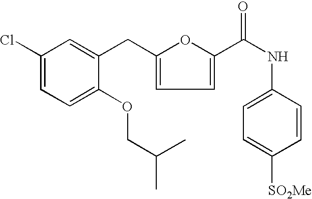 | 5-({5-Chloro-2-[(2-methylpropyl)oxy]-phenyl}methyl)-N-[4-(methylsulfonyl)-phenyl]-2-furancarboxamide | LC/MSRt = 3.39[MH]⁺ 462.0,464.0 |
| 60 | 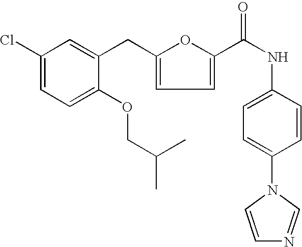 | 5-({5-Chloro-2-[(2-methylpropyl)oxy]-phenyl}methyl)-N-[4-(1H-imidazol-1-yl)phenyl]-2-furancarboxamide | LC/MSRt = 2.59[MH]⁺ 450.1,452.1 |
| 61 | 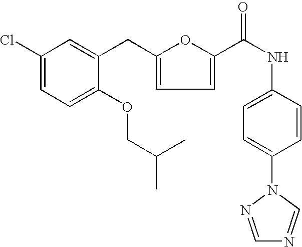 | 5-({5-Chloro-2-[(2-methylpropyl)oxy]-phenyl}methyl)-N-[4-(1H-1,2,4-triazol-1-yl)phenyl]-2-furancarboxamide | LC/MSRt = 3.42[MH]⁺ 451.0,453.1 |
| 62 | 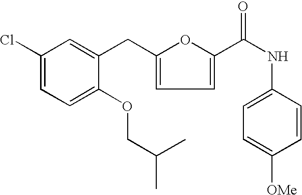 | 5-({5-Chloro-2-[(2-methylpropyl)oxy]-phenyl}methyl)-N-[4-(methyloxy)phenyl]-2-furancarboxamide | LC/MSRt = 3.55[MH]⁺ 414.1 |
| 63 | 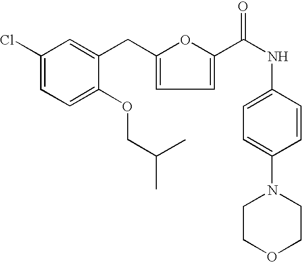 | 5-({5-Chloro-2-[(2-methylpropyl)oxy]-phenyl}methyl)-N-[4-(4-morpholinyl)phenyl]-2-furancarboxamide | LC/MSRt = 3.51[MH]⁺ 469.1,471.1 |
| 64 | 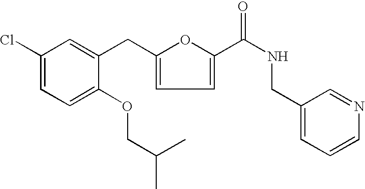 | 5-({5-Chloro-2-[(2-methylpropyl)oxy]-phenyl}methyl)-N-(3-pyridinylmethyl)-2-furancarboxamide | LC/MSRt = 2.79[MH]⁺ 399.2,401.2 |
| 65 | 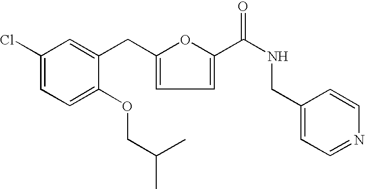 | 5-({5-Chloro-2-[(2-methylpropyl)oxy]-phenyl}methyl)-N-(4-pyridinylmethyl)-2-furancarboxamide | LC/MSRt = 2.61[MH]⁺ 399.2 |
| 66 | 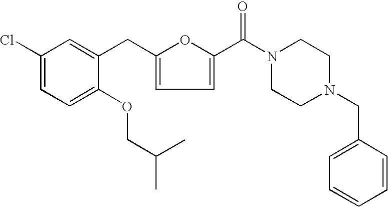 | 1-{[5-({5-Chloro-2-[(2-methylpropyl)oxy]-phenyl}methyl)-2-furanyl]carbonyl}-4-(phenylmethyl)-piperazine | LC/MSRt = 2.51[MH]⁺ 467.2,469.2 |
| 67 | 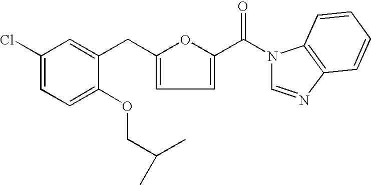 | 1-{[5-({5-Chloro-2-[(2-methylpropyl)oxy]-phenyl}methyl)-2-furanyl]carbonyl}-1H-benzimidazole | LC/MSRt = 3.91[MH]⁺ 409.0 |
| 68 | 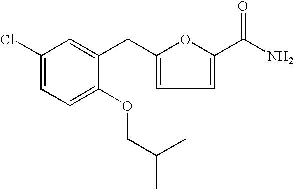 | 5-({5-Chloro-2-[(2-methylpropyl)oxy]-phenyl}methyl)-2-furancarboxamide | LC/MSRt = 3.15[MH]⁺ 308.2,310.2 |
| 69 | 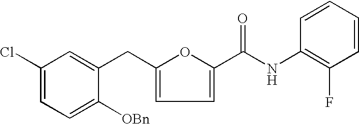 | 5-({5-Chloro-2-[(phenylmethyl)oxy]-phenyl}methyl)-N-(2-fluorophenyl)-2-furancarboxamide | LC/MSRt = 3.65[MH]⁺ 436.2,438.2 |
| 70 | 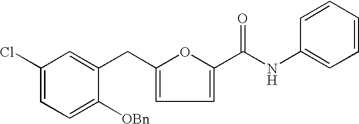 | 5-({5-Chloro-2-[(phenylmethyl)oxy]-phenyl}methyl)-N-phenyl-2-furancarboxamide | LC/MSRt = 3.61[MH]⁺ 418.2,420.2 |
| 71 | 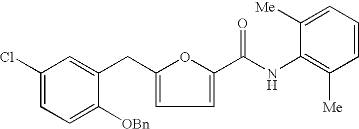 | 5-({5-Chloro-2-[(phenylmethyl)oxy]-phenyl}methyl)-N-(2,6-dimethylphenyl)-2-furancarboxamide | LC/MSRt = 3.59[MH]⁺ 446.2,448.2 |
| 72 | 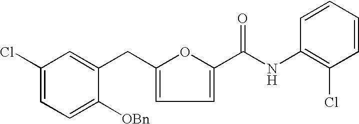 | N-(2-chlorophenyl)-5-({5-chloro-2-[(phenylmethyl)oxy]-phenyl}methyl)-2-furancarboxamide | LC/MSRt = 3.85[MH]⁺ 452.2 |
| 73 | 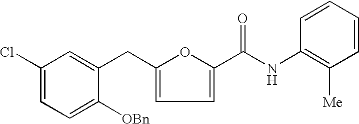 | 5-({5-Chloro-2-[(phenylmethyl)oxy]-phenyl}methyl)-N-(2-methylphenyl)-2-furancarboxamide | LC/MSRt = 3.63[MH]⁺ 432.2,434.2 |
| 74 | 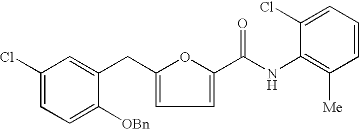 | N-(2-Chloro-6-methylphenyl)-5-({5-chloro-2-[(phenylmethyl)oxy]-phenyl}methyl)-2-furancarboxamide | LC/MSRt = 3.58[MH]⁺ 466.2 |
| 75 | 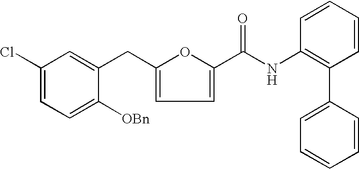 | N-2-biphenylyl-5-({5-chloro-2-[(phenylmethyl)oxy]-phenyl}methyl)-2-furancarboxamide | LC/MSRt = 3.90[MH]⁺ 494.3,496.2 |
| 76 | 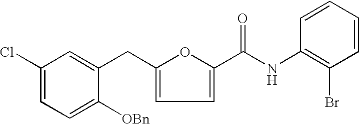 | N-(2-bromophenyl)-5-({5-chloro-2-[(phenylmethyl)oxy]-phenyl}methyl)-2-furancarboxamide | LC/MSRt = 3.90[MH]⁺ 498.1,500.1 |
| 77 | 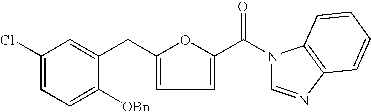 | 1-{[5-({5-Chloro-2-[(phenylmethyl)oxy]-phenyl}methyl)-2-furanyl]carbonyl}-1H-benzimidazole | LC/MSRt = 3.80[MH]⁺ 443.0,445.0 |
| 78 | 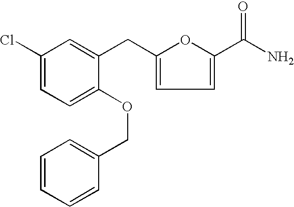 | 5-({5-Chloro-2-[(phenylmethyl)oxy]-phenyl}methyl)-2-furancarboxamide | LC/MSRt = 3.07[MH]⁺ 342.2,344.2 |
| 79 | 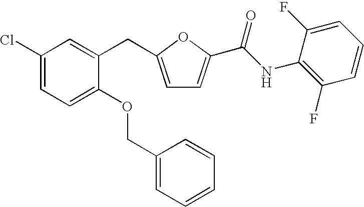 | 5-({5-Chloro-2-[(phenylmethyl)oxy]-phenyl}methyl)-N-(2,6-difluorophenyl)-2-furancarboxamide | LC/MS Rt =3.38 min,[MH⁺] 454,456. |
| 80 | 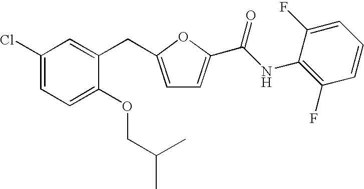 | 5-({5-Chloro-2-[(2-methylpropyl)oxy]-phenyl}methyl)-N-(2,6-difluorophenyl)-2-furancarboxamide | LC/MS Rt =3.72 min,[MH⁺] 420,422. |

### 5-[(5-Chloro-2-hydroxyphenyl)methyl]-2-furancarboxylic acid

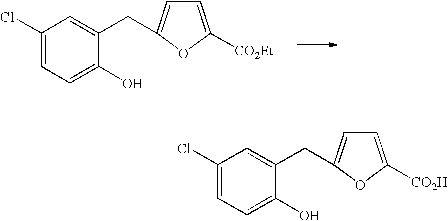

A solution of ethyl 5-[(5-chloro-2-hydroxyphenyl)methyl]-2-furancarboxylate in ethanol (30 ml) and 2M sodium hydroxide (20 ml) was left at room temperature overnight and evaporated to dryness. The residue was dissolved in water/diethyl ether and the aqueous layer separated, acidified with concentrated hydrochloric acid and extracted with ether before being dried (magnesium sulphate) and evaporated to give the title compound as a light brown solid (2.41 g). LC/MS: Rt=2.37, [MH]⁻ 250.8, 252.8.

### 5-{[2-(Acetyloxy)-5-chlorophenyl]methyl}-2-furancarboxylic acid

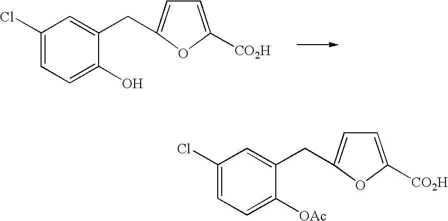

Acetic anhydride (5 ml) was added to a solution of 5-[(5-chloro-2-hydroxyphenyl)methyl]-2-furancarboxylic acid in pyridine (10 ml) and the mixture heated at 80° C. for 30 minutes then cooled and evaporated to dryness. The residue was dissolved in diethyl ether, washed with 2M hydrochloric acid, dried (magnesium sulphate) and evaporated then re-evaporated with toluene to give the title compound as a yellow gum (2.74 g).

LC/MS: Rt=2.96, [MH]⁺ 295.1, 297.1

### 4-Chloro-2-[(5-{[(2,6-difluorophenyl)amino]carbonyl}-2-furanyl)methyl]phenyl acetate

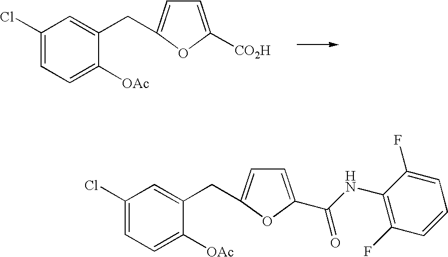

Oxalyl chloride (5 ml) was added to a solution of 5-{[2-(acetyloxy)-5-chlorophenyl]methyl}-2-furancarboxylic acid (2.72 g, 9.24 mmol) in dichloromethane (25 ml) and one drop of dimethylformamide, left at room temperature for 1.5 hours and evaporated to dryness. The residue was dissolved in dichloromethane (10 ml) and added over 5 minutes to a solution of 2,6-difluoroaniline (1.935 g, 15 mmol) in dichloromethane (15 ml) and pyridine (5 ml). After 30 minutes at room temperature the solution was washed with 2M hydrochloric acid, dried (magnesium sulphate), evaporated to dryness and the residue purified by chromatography on silica gel to give the title compound as a white solid after trituration with diethyl ether (3.41 g). LC/MS: Rt=3.04, [MH]⁺ 406.1, 408.1.

### 5-[(5-Chloro-2-hydroxyphenyl)methyl]-N-(2,6-difluorophenyl)-2-furancarboxamide

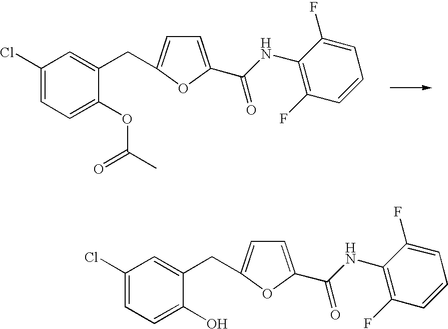

4-Chloro-2-[(5-{[(2,6-difluorophenyl)amino]carbonyl}-2-furanyl)methyl]phenyl acetate (3.4 g, 8.38 mmol) was dissolved in ethanol (40 ml) and 2M sodium hydroxide (10 ml), left at room temperature for 18 hours and evaporated to dryness. The residue was dissolved in diethyl ether/water, acidified with 2M hydrochloric acid and the organic phase dried (magnesium sulphate), evaporated to dryness and the residue triturated with diethyl ether/hexane to give the title compound as a white solid (2.91 g). LC/MS: Rt=2.82, [MH]⁺ 364.0, 366.0.

### Example 81

### 5-[(5-Chloro-2-{[(2-fluorophenyl)methyl]oxy}phenyl)methyl]-N-(2,6-difluorophenyl)-2-furancarboxamide

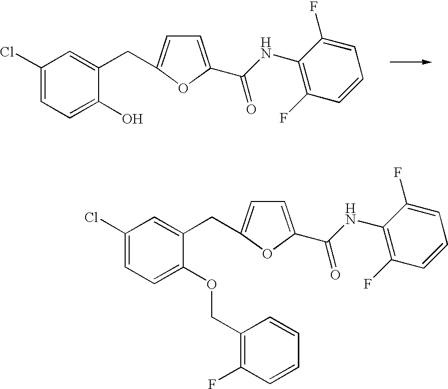

A mixture of 5-[(5-chloro-2-hydroxyphenyl)methyl]-N-(2,6-difluorophenyl)-2-furancarboxamide (121 mg, 0.33 mmol), 2-fluorobenzyl bromide (76 mg, 0.4 mmol) and potassium carbonate (138 mg, 1 mmol) in acetone (4 ml) was stirred and refluxed for 2 hours. After cooling the mixture was filtered, evaporated, purified by chromatography on silica gel eluting with ethyl acetate/hexane (1:4) and triturated with diethyl ether/hexane to give the title compound as a white solid (91 mg). LC/MS: Rt=3.57, [MH]⁺ 472.0, 474.0.

The following examples were prepared in a similar manner to 5-[(5-chloro-2-{[(2-fluorophenyl)methyl]oxy}phenyl)methyl]-N-(2,6-difluorophenyl)-2-furancarboxamide from the appropriate intermediates.

| Example | Structure | Name | Data |
|---|---|---|---|
| 82 | 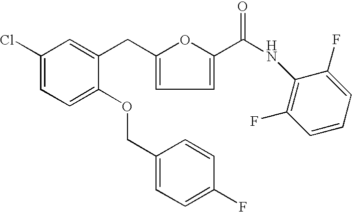 | 5-[(5-Chloro-2-{[(4-fluorophenyl)methyl]oxy}-phenyl)methyl]-N-(2,6-difluorophenyl)-2-furancarboxamide | LC/MS Rt = 3.55[MH]⁺ 472.0 |
| 83 | 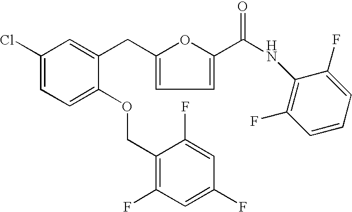 | 5-[(5-Chloro-2-{[(2,4,6-trifluorophenyl)methyl]oxy}-phenyl)methyl]-N-(2,6-difluorophenyl)-2-furancarboxamide | LC/MS Rt = 3.58[MH]⁺ 508.0,510.0 |
| 84 | 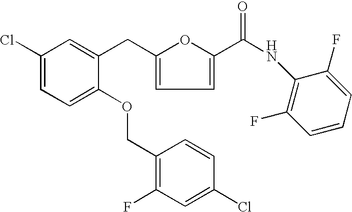 | 5-[(5-Chloro-2-{[(4-chloro-2-fluorophenyl)methyl]oxy}-phenyl)methyl]-N-(2,6-difluorophenyl)-2-furancarboxamide | LC/MS Rt = 3.75[MH]⁺ 506.1 |
| 85 | 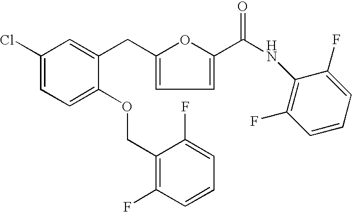 | 5-[(5-Chloro-2-{[(2,6-difluorophenyl)methyl]oxy}-phenyl)methyl]-N-(2,6-difluorophenyl)-2-furancarboxamide | LC/MS Rt = 3.57[MH]⁺ 490.1,492.1 |
| 86 | 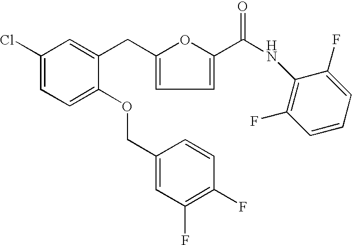 | 5-[(5-Chloro-2-{[(3,4-difluorophenyl)methyl]oxy}-phenyl)methyl]-N-(2,6-difluorophenyl)-2-furancarboxamide | LC/MS Rt = 3.59[MH]⁺ 490.1,492.0 |
| 87 | 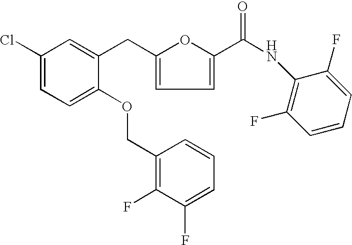 | 5-[(5-Chloro-2-{[(2,3-difluorophenyl)methyl]oxy}-phenyl)methyl]-N-(2,6-difluorophenyl)-2-furancarboxamide | LC/MS Rt = 3.60[MH]⁺ 490.0,492.1 |
| 88 | 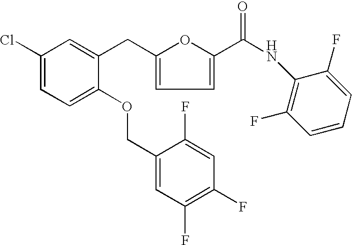 | 5-[(5-Chloro-2-{[(2,4,5-trifluorophenyl)methyl]oxy}-phenyl)methyl]-N-(2,6-difluorophenyl)-2-furancarboxamide | LC/MS Rt = 3.62[MH]⁺ 508.1,510.0 |
| 89 | 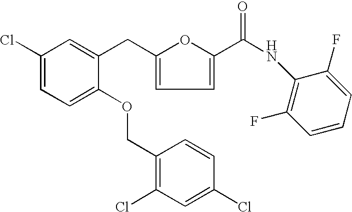 | 5-[(5-Chloro-2-{[(2,4-dichlorophenyl)methyl]-oxy}phenyl)methyl]-N-(2,6-difluorophenyl)-2-furancarboxamide | LC/MS Rt = 3.92[MH]⁺ 524.0,526.0 |
| 90 | 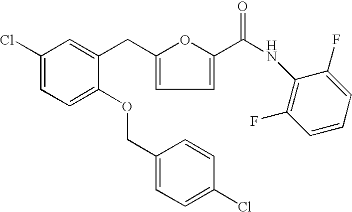 | 5-[(5-Chloro-2-{[(4-chlorophenyl)methyl]oxy}-phenyl)methyl]-N-(2,6-difluorophenyl)-2-furancarboxamide | LC/MS Rt = 3.68[MH]⁺ 488.1 |

### Example 91

### 5-({5-Chloro-2-[(2-pyridinylmethyl)oxy]phenyl}methyl)-N-(2,6-difluorophenyl)-2-furancarboxamide

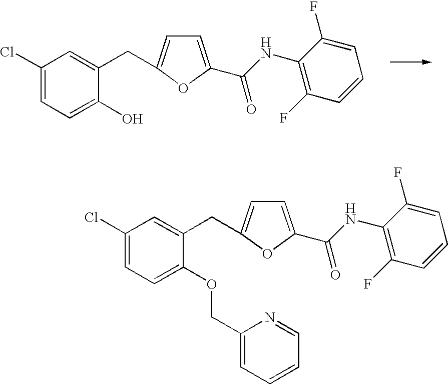

A mixture of 5-[(5-chloro-2-hydroxyphenyl)methyl]-N-(2,6-difluorophenyl)-2-furancarboxamide (182 mg, 0.5 mmol), 2-picolyl chloride hydrochloride (90 mg, 0.55 mmol) and potassium carbonate (276 mg, 2 mmol) in dimethylformamide (3 ml) was stirred and heated at 80° C. for 3 hours. After cooling the mixture was diluted with diethyl ether/water and the organic phase washed twice with water before being dried (magnesium sulphate), evaporated, purified by chromatography on silica gel eluting with ethyl acetate/hexane (2:1) and triturated with diethyl ether/hexane to give the title compound as a white solid (46 mg). LC/MS: Rt=3.05, [MH]⁺ 455.2.

The following compounds were prepared by the above method.

| Example | Structure | Name | Data |
|---|---|---|---|
| 92 | 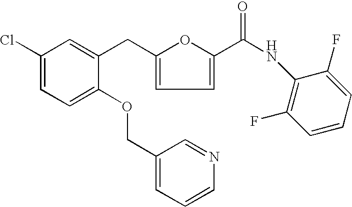 | 5-({5-Chloro-2-[(3-pyridinylmethyl)oxy]phenyl}methyl)-N-(2,6-difluorophenyl)-2-furancarboxamide | LC/MSRt = 2.62[MH]⁺ 455.2 |
| 93 | 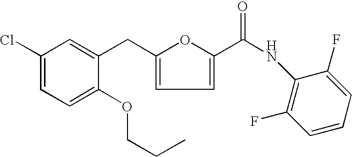 | 5-{[5-Chloro-2-(propyloxy)phenyl]methyl}-N-(2,6-difluorophenyl)-2-furancarboxamide | LC/MSRt = 3.51[MH]⁺ 406.2,408.2 |
| 94 | 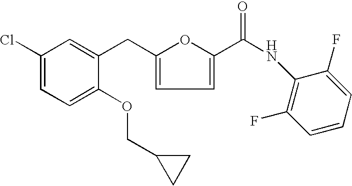 | 5-({5-Chloro-2-[(cyclopropylmethyl)oxy]-phenyl}methyl)-N-(2,6-difluorophenyl)-2-furancarboxamide | LC/MSRt = 3.49[MH]⁺ 418.2,420.2 |
| 95 | 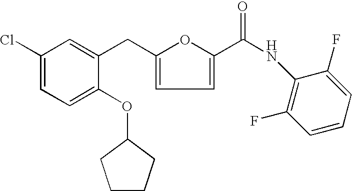 | 5-{[5-Chloro-2-(cyclopentyloxy)phenyl]-methyl}-N-(2,6-difluorophenyl)-2-furancarboxamide | LC/MSRt = 3.67[MH]⁺ 432.2,434.2 |
| 96 | 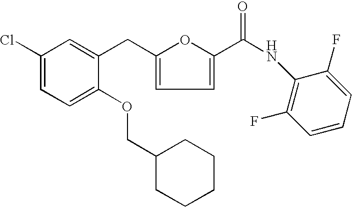 | 5-({5-Chloro-2-[(cyclohexylmethyl)oxy]-phenyl}methyl)-N-(2,6-difluorophenyl)-2-furancarboxamide | LC/MSRt = 3.96[MH]⁺ 460.3,462.2 |
| 97 | 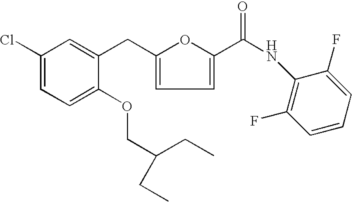 | 5-({5-Chloro-2-[(2-ethylbutyl)oxy]phenyl}meth-yl)-N-(2,6-difluorophenyl)-2-furancarboxamide | LC/MSRt = 3.89[MH]⁺ 448.2,450.2 |

### 5-({5-Chloro-2-[(phenylmethyl)oxy]phenyl}methyl)-2-furancarbonitrile

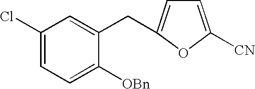

A solution of 5-({5-chloro-2-[(phenylmethyl)oxy]phenyl}methyl)-2-furancarboxamide (200 mg, 0.59 mmol) in phosphoryl chloride (1 ml) was stirred and heated at 80° C. for 1 hour then poured onto ice and diluted with diethyl ether/2M sodium hydroxide. The organic phase was dried (magnesium sulphate), evaporated and purified by chromatography on silica gel eluting with ethyl acetate/hexane (1:19) to give the title compound as a colourless gum (98 mg).

¹H NMR (CDCl₃) δ: 4.0 (s, 2H), 5.04 (s, 2H), 6.06 (d, 1H), 6.85 (d, 1H), 6.98 (d, 1H), 7.12 (d, 1H), 7.19 (dd, 1H), 7.31-7.40 (m, 5H).

### 5-({5-Chloro-2-[(2-methylpropyl)oxy]phenyl}methyl)-2-furancarbonitrile

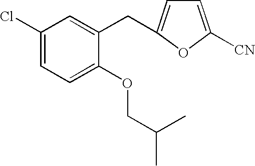

Prepared by the same method as for 5-({5-chloro-2-[(phenylmethyl)oxy]phenyl}methyl)-2-furancarbonitrile but using 5-({5-chloro-2-[(2-methylpropyl)oxy]phenyl}methyl)-2-furancarboxamide instead of 5-({5-chloro-2-[(phenylmethyl)oxy]phenyl}methyl)-2-furancarboxamide. LC/MS: Rt=3.66, [MH]⁺ 304.3.

### Methyl 5-({5-chloro-2-[(phenylmethyl)oxy]phenyl}methyl)-2-furancarboximidoate hydrochloride

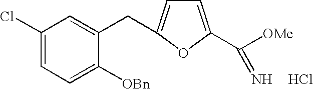

60% Sodium hydride (40 mg, 1 mmol) was added to a solution 5-({5-chloro-2-[(phenylmethyl)oxy]phenyl}methyl)-2-furancarbonitrile (4 g, 12.35 mmol) in methanol (50 ml) and the solution left at room temperature for 18 hours then evaporated to dryness. The residue was dissolved in diethyl ether, washed with water and the organic phase dried (magnesium sulphate), filtered and 1M hydrogen chloride in ether (20 ml) added. The precipitate was filtered off to give the title compound as a white solid (4.3 g).

LC/MS: Rt=2.57, [MH]⁺ 356.0

### Methyl 5-({5-chloro-2-[(2-methylpropyl)oxy]phenyl}methyl)-2-furancarboximidoate hydrochloride

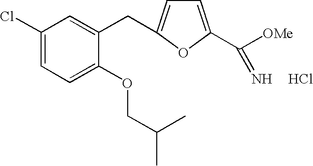

Prepared as for methyl 5-({5-chloro-2-[(phenylmethyl)oxy]phenyl}methyl)-2-furancarboximidoate hydrochloride but using 5-({5-chloro-2-[(2-methylpropyl)oxy]phenyl}methyl)-2-furancarbonitrile instead of 5-({5-chloro-2-[(phenylmethyl)oxy]phenyl}methyl)-2-furancarbonitrile. LC/MS: Rt=2.64, [MH]⁺ 322.1.

### Example 98

### 2-[5-({5-Chloro-2-[(2-methylpropyl)oxy]phenyl}methyl)-2-furanyl]-1H-benzimidazole

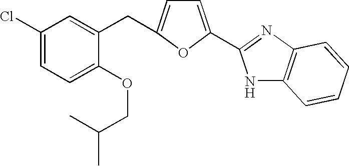

A mixture of 1,2 phenylenediamine (37 mg, 0.34 mmol) and methyl 5-({5-chloro-2-[(2-methylpropyl)oxy]phenyl}methyl)-2-furancarboximidoate hydrochloride (124 mg, 0.33 mmol) in ethanol (3 ml) was stirred and refluxed for 3 hours. The resulting mixture was diluted with diethyl ether/2M sodium hydroxide, the organic phase dried (magnesium sulphate), evaporated and the residue purified by chromatography on silica gel eluting with ethyl acetate/hexane (15:85). The product was triturated with diethyl ether to give the title compound as a white solid (91 mg). LC/MS: Rt=2.90, [MH]⁺ 381.1.

The following compounds were prepared by a similar procedure to above by heating either ethyl 5-({5-chloro-2-[(2-methylpropyl)oxy]phenyl}methyl)-2-furancarboximidoate or methyl 5-({5-chloro-2-[(phenylmethyl)oxy]phenyl}methyl)-2-furancarboximidoate hydrochloride with the appropriate diamine in ethanol.

| Example | Structure | Name | Data |
|---|---|---|---|
| 99 | 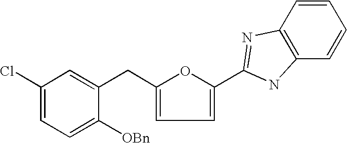 | 2-[5-({5-Chloro-2-[(phenylmethyl)oxy]phenyl}methyl)-2-furanyl]-1H-benzimidazole | LC/MSRt = 2.84[MH]⁺ 415.1 |
| 100 | 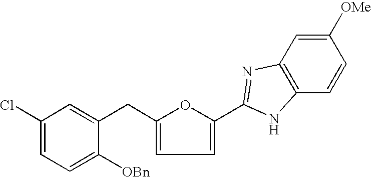 | 2-[5-({5-Chloro-2-[(phenylmethyl)oxy]phenyl}methyl)-2-furanyl]-5-(methyloxy)-1H-benzimidazole | LC/MSRt = 2.73[MH]⁺ 445.0,447.0 |
| 101 | 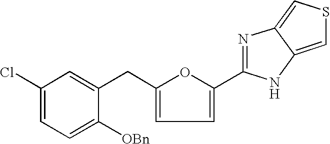 | 2-[5-({5-Chloro-2-[(phenylmethyl)oxy]phenyl}methyl)-2-furanyl]-1H-thieno[3,4-d]imidazole | LC/MSRt = 2.89[MH]⁺ 421.0 |
| 102 | 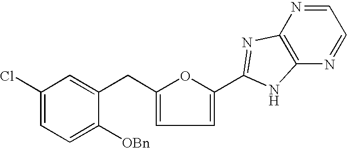 | 2-[5-({5-Chloro-2-[(phenylmethyl)oxy]phenyl}methyl)-2-furanyl]-1H-imidazo[4,5-b]pyrazine | LC/MSRt = 3.06[MH]⁺ 417.0,419.9 |
| 103 | 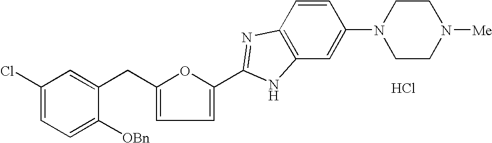 | 2-[5-({5-Chloro-2-[(phenylmethyl)oxy]phenyl}methyl)-2-furanyl]-5-(4-methyl-1-piperazinyl)-1H-benzimidazolehydrochloride | LC/MSRt = 2.08[MH]⁺ 513.3 |
| 104 | 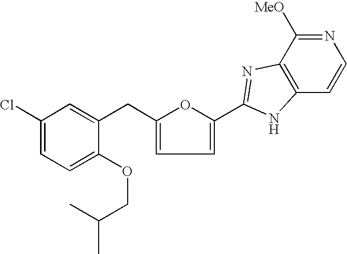 | 2-[5-({5-Chloro-2-[(2-methylpropyl)oxy]-phenyl}methyl)-2-furanyl]-4-(methyloxy)-1H-imidazo[4,5-c]pyridine | LC/MSRt = 3.28[MH]⁺ 412.2 |
| 105 | 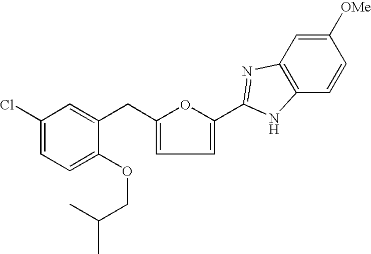 | 2-[5-({5-Chloro-2-[(2-methylpropyl)oxy]-phenyl}methyl)-2-furanyl]-5-(methyloxy)-1H-benzimidazole | LC/MSRt = 2.81[MH]⁺ 411.2 |
| 106 | 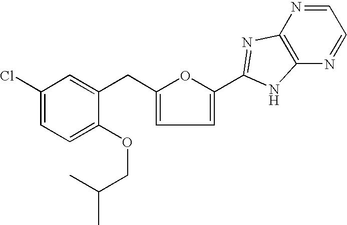 | 2-[5-({5-Chloro-2-[(2-methylpropyl)oxy]-phenyl}methyl)-2-furanyl]-1H-imidazo[4,5-b]pyrazine | LC/MSRt = 3.15[MH]⁺ 383.1,385.1 |
| 107 | 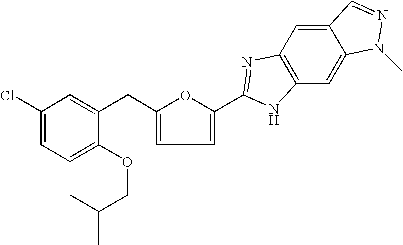 | 6-[5-({5-Chloro-2-[(2-methylpropyl)oxy]-phenyl}methyl)-2-furanyl]-1-methyl-1,5-dihydroimidazo[4,5-f]indazole | LC/MSRt = 2.84[MH]⁺ 435.2 |
| 108 | 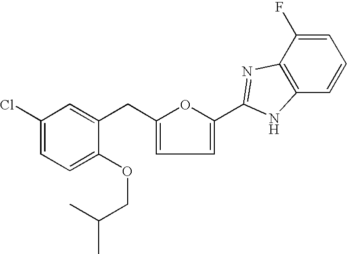 | 2-[5-({5-Chloro-2-[(2-methylpropyl)oxy]-phenyl}methyl)-2-furanyl]-4-fluoro-1H-benzimidazole | LC/MSRt = 3.59[MH]⁺ 399.0 |
| 109 | 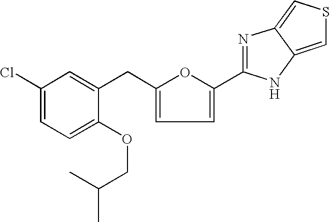 | 2-[5-({5-Chloro-2-[(2-methylpropyl)oxy]-phenyl}methyl)-2-furanyl]-1H-thieno[3,4-d]imidazole | LC/MSRt = 2.99[MH]⁺ 387.1 |
| 110 | 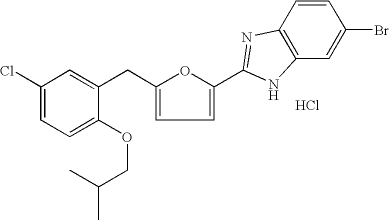 | 5-Bromo-2-[5-({5-chloro-2-[(2-methylpropyl)oxy]-phenyl}methyl)-2-furanyl]-1H-benzimidazolehydrochloride | LC/MSRt = 3.77[MH]⁺ 461.0,462.9 |
| 111 | 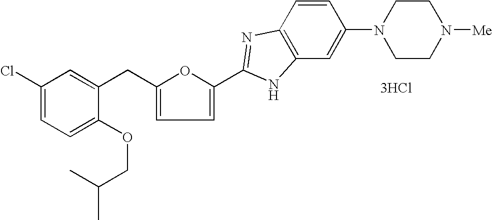 | 2-[5-({5-Chloro-2-[(2-methylpropyl)oxy]-phenyl}methyl)-2-furanyl]-5-(4-methyl-1-piperazinyl)-1H-benzimidazoletrihydrochloride | LC/MSRt = 2.10[MH]⁺ 479.3,481.3 |
| 112 | 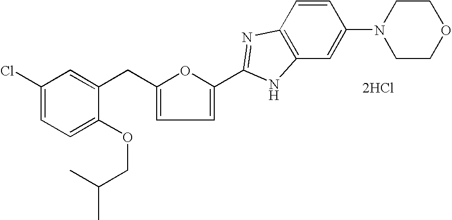 | 2-[5-({5-Chloro-2-[(2-methylpropyl)oxy]-phenyl}methyl)-2-furanyl]-5-(4-morpholinyl)-1H-benzimidazoledihydrochloride | LC/MSRt = 2.66[MH]⁺ 466.2,468.3 |
| | 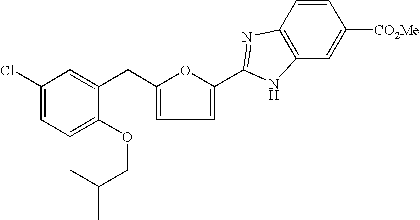 | Methyl 2-[5-({5-chloro-2-[(2-methylpropyl)oxy]-phenyl}methyl)-2-furanyl]-1H-benzimidazole-5-carboxylate | LC/MSRt = 3.53[MH]⁺ 439.2,441.2 |
| 113 | 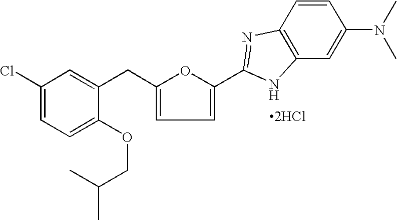 | 2-[5-({5-Chloro-2-[(2-methylpropyl)oxy]-phenyl}methyl)-2-furanyl]-N,N-dimethyl-1H-benzimidazol-5-aminedihydrochloride | LC/MSRt = 2.57[MH]⁺ 424.3,426.3 |
| 114 | 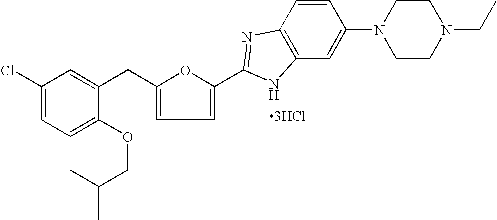 | 2-[5-({5-Chloro-2-[(2-methylpropyl)oxy]-phenyl}methyl)-2-furanyl]-5-(4-ethyl-1-piperazinyl)-1H-benzimidazoletrihydrochloride | LC/MSRt = 2.19[MH]⁻499.22,493.23 |
| 115 | 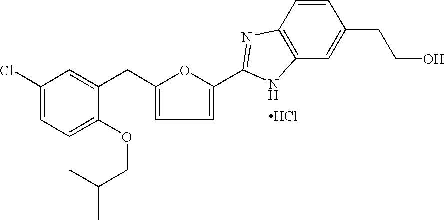 | 2-{2-[5-({5-Chloro-2-[(2-methylpropyl)oxy]-phenyl}methyl)-2-furanyl]-1H-benzimidazol-5-yl}ethanolhydrochloride | LC/MSRt = 2.55[MH]⁻423.09,425.08 |
| | 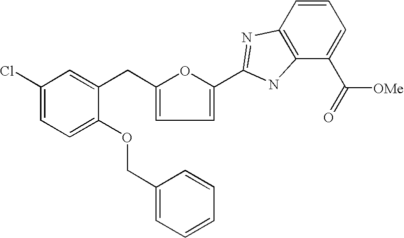 | Methyl 2-[5-({5-chloro-2-[(phenylmethyl)oxy]phenyl}methyl)-2-furanyl]-1H-benzimidazole-4-carboxylate | LC/MS. Rt =3.79 min.[M + H] 4391 Cl. |

The following compounds were prepared as above but using acetic acid as solvent instead of ethanol.

| Examples | Structure | Name | LC/MS |
|---|---|---|---|
| 116 | 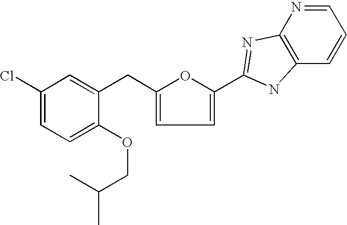 | 2-[5-({5-Chloro-2-[(2-methylpropyl)oxy]phenyl}-methyl)-2-furanyl]-1H-imidazo[4,5-b]pyridine | LC/MSRt = 3.01[MH]⁺ 382.1 |
| 117 | 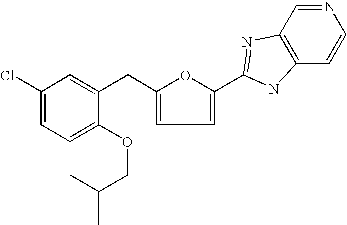 | 2-[5-({5-Chloro-2-[(2-methylpropyl)oxy]phenyl}-methyl)-2-furanyl]-1H-imidazo[4,5-c]pyridine | LC/MSRt = 2.32[MH]⁺ 382.1 |
| 118 | 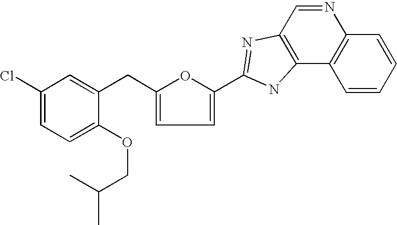 | 2-[5-({5-Chloro-2-[(2-methylpropyl)oxy]phenyl}-methyl)-2-furanyl]-1H-imidazo[4,5-c]quinoline | LC/MSRt = 2.65[MH]⁺ 432.1 |
| 119 | 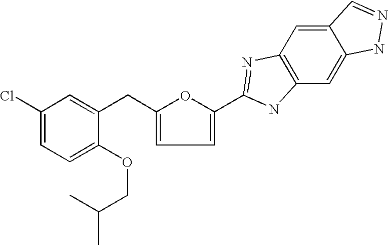 | 6-[5-({5-Chloro-2-[(2-methylpropyl)oxy]phenyl}-methyl)-2-furanyl]-1,5-dihydroimidazo[4,5-f]indazole | LC/MSRt = 2.65[MH]⁺ 421.1 |
| 120 | 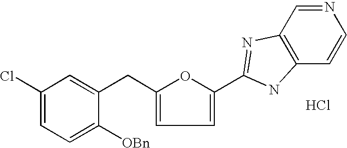 | 2-[5-({5-Chloro-2-[(phenylmethyl)oxy]-phenyl}methyl)-2-furanyl]-1H-imidazo[4,5-c]pyridine hydrochloride | LC/MSRt = 2.31[MH]⁺416.1,418.1 |
| 121 | 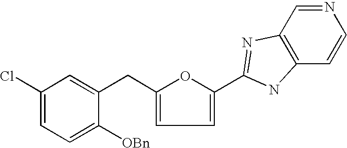 | 2-[5-({5-Chloro-2-[(phenylmethyl)oxy]-phenyl}methyl)-2-furanyl]-1H-imidazo[4,5-b]pyridine | LC/MSRt = 2.93[MH]⁺416.1,418.1 |

### 2-(3,4-Diaminophenyl)ethanol

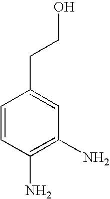

Zinc dust (13.63 g, 208.8 mmol) was added in portions to a stirred solution of 2-(4-amino-3-nitrophenyl)ethanol (3.8 g, 20.88 mmol) in acetic acid (80 ml) with water bath cooling and the mixture stirred for one hour then filtered and evaporated. The residue was re-evaporated with toluene, dissolved in methanol and triethylamine (20 ml) added. The solution was evaporated and purified by flash chromatography on silica eluting with methanol/dichloromethane (8:92) and triturated with ether to give the title compound as an off-white solid which darkened on standing. LC/MS Rt=0.32 min, [MH⁺] 153.08.

### [5-(4-Ethyl-1-piperazinyl)-2-nitrophenyl]amine

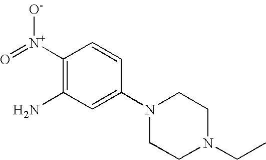

5-Chloro-2-nitroaniline (3 g, 17.4 mmol), 1-ethylpiperazine (4.42 ml, 34.9 mmol) and potassium carbonate (2.4 g, 17.4 mmol) in DMF (8 ml) were heated at 130° C. for 2 h in a Smithcreator® microwave. The mixture was diluted with water and ethyl acetate. A yellow solid formed, it was filtered off and dried to give 1.3 g of the title compound.

LC/MS Rt=1.21, [MH⁺] 251.2, 252.2

### 4-(4-Ethyl-1-piperazinyl)-1,2-benzenediamine

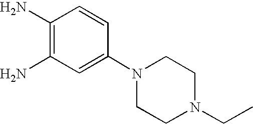

A solution of [5-(4-ethyl-1-piperazinyl)-2-nitrophenyl]amine (1.3 g, 5.2 mmol) in ethanol (140 ml) was hydrogenated with ˜150 mg of Pd/C in ethanol (140 ml) for 18 h.

The catalyst was filtered off and the filtrate evaporated to give the titled compound as a black solid (1.07 g). LC/MS Rt=0.31, [MH⁺] 221.3, 222.3.

### [5-({5-Chloro-2-[(2-methylpropyl)oxy]phenyl}methyl)-2-furanyl]methanol

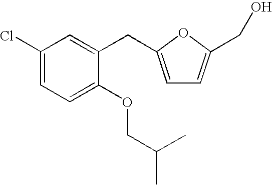

Ethyl 5-({5-chloro-2-[(2-methylpropyl)oxy]phenyl}methyl)-2-furancarboxylate (3.37 g, 10 mmol) was dissolved in tetrahydrofuran (30 ml) and cooled to 0° C. under argon with stirring. 1M Lithium aluminium hydride in diethyl ether (10 ml, 10 mmol) was added at 0-5° C. over 10 minutes and the reaction stirred at 0° C. for 1 h. Water (5 ml) was carefully added. When effervescence had ceased, diethyl ether and water were added and the organic layer washed with brine, dried (MgSO₄) and evaporated. The residue was purified by flash chromatography, eluting with 5-30% ethyl acetate in hexane to give the title compound as a yellow oil (2.58 g).

¹H NMR (CDCl₃) δ: 1.00 (6H, d), 1.65 (1H, t), 2.07 (1H, m), 3.69 (2H, d), 3.94 (2H, s), 4.55 (2H, d), 5.94 (1H, d), 6.19 (1H, d), 6.74 (1H, d), 7.08 (1H, d), 7.14 (1H, dd).

### [5-({5-Chloro-2-[(phenylmethyl)oxy]phenyl}methyl)-2-furanyl]methanol

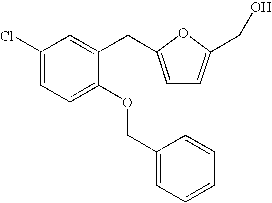

The title compound was prepared in a similar manner to [5-({5-chloro-2-[(2-methylpropyl)oxy]phenyl}methyl)-2-furanyl]methanol using ethyl 5-({5-chloro-2-[(2-phenylmethyl)oxy]phenyl}methyl)-2-furancarboxylate.

LC/MS Rt=3.38 min, [MH] 327, 329.

### 5-({5-Chloro-2-[(2-methylpropyl)oxy]phenyl}methyl)-2-furancarbaldehyde

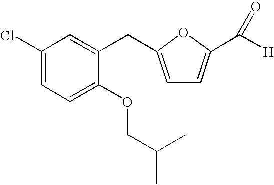

[5-({5-Chloro-2-[(2-methylpropyl)oxy]phenyl}methyl)-2-furanyl]methanol (884 mg, 3 mmol) was dissolved in dichloromethane (15 ml) and Dess-Martin periodinane (1.4 g, 3.3 mmol) added. The mixture was stirred at room temperature for 2 h then washed with 5% sodium thiosulphate solution and water, dried (MgSO₄) and evaporated. The residue was purified by flash chromatography, eluting with 5-30% ethyl acetate in hexane to give the title compound as a yellow oil (592 mg). LC/MS Rt=3.56 min, [MH] 291, 293.

### 5-({5-Chloro-2-[(phenylmethyl)oxy]phenyl}methyl)-2-furancarbaldehyde

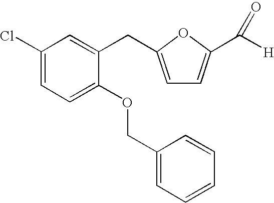

The title compound was prepared in a similar manner to 5-({5-chloro-2-[(2-methylpropyl)oxy]phenyl}methyl)-2-furancarbaldehyde using [5-({5-chloro-2-[(phenylmethyl)oxy]phenyl}methyl)-2-furanyl]methanol.

LC/MS Rt=3.49 min, [MH⁺] 327, 329.

### Example 122

### 2-[5-({5-Chloro-2-[(2-methyipropyl)oxy]phenyl}methyl)-2-furanyl]-5-fluoro-1H-benzimidazole

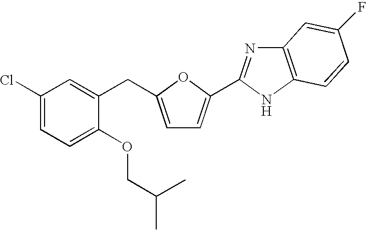

5-({5-Chloro-2-[(2-methylpropyl)oxy]phenyl}methyl)-2-furancarbaldehyde (100 mg, 0.33 mmol) was dissolved in methanol (1 ml) and added dropwise to a suspension of sodium hydrogen sulphite (39 mg, 0.38 mmol) and 4-fluoro-1,2-phenylenediamine (43 mg, 0.33 mmol) in methanol (1 ml). The mixture was heated to reflux for 1 h, filtered, washed with methanol and evaporated. The residue was dissolved in ethyl acetate and washed with 0.5M hydrochloric acid, 5% sodium bicarbonate solution and water, dried (MgSO₄) and evaporated. The residue was purified by flash chromatography, eluting with 5-30% ethyl acetate in hexane and the product triturated with diethyl ether, filtered and dried in vacuo (31 mg). LC/MS Rt=3.36 min, [MH⁺] 399, 401.

The following examples were prepared in a similar manner to 2-[5-({5-chloro-2-[(2-methylpropyl)oxy]phenyl}methyl)-2-furanyl]-5-fluoro-1H-benzimidazole from the appropriate intermediates.

| Example | | Name | Data |
|---|---|---|---|
| 123 | 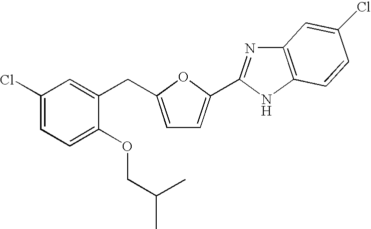 | 5-Chloro-2-[5-({5-chloro-2-[(2-methylpropyl)oxy]phenyl}-methyl)-2-furanyl]-1H-benzimidazole | LC/MS Rt = 3.62min, [MH⁺] 415,418. |
| 124 | 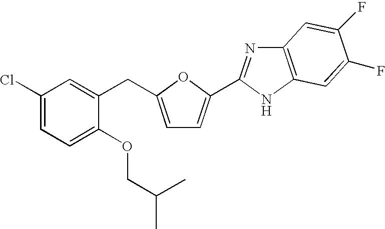 | 2-[5-({5-Chloro-2-[(2-methylpropyl)oxy]phenyl}-methyl)-2-furanyl]-5,6-difluoro-1H-benzimidazole | LC/MS Rt = 3.70min, [MH⁺] 417,419. |
| 125 | 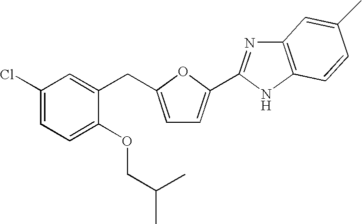 | 2-[5-({5-Chloro-2-[(2-methylpropyl)oxy]phenyl}-methyl)-2-furanyl]-5-methyl-1H-benzimidazole | LC/MS Rt = 2.90min, [MH⁺] 395,397. |
| 126 | 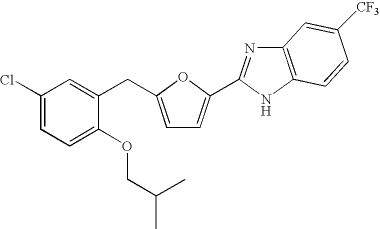 | 2-[5-({5-Chloro-2-[(2-methylpropyl)oxy]phenyl}-methyl)-2-furanyl]-5-(trifluoromethyl)-1H-benzimidazole | LC/MS Rt = 3.87min, [MH⁺] 449,451. |
| 127 | 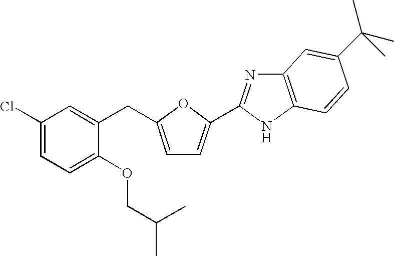 | 2-[5-({5-Chloro-2-[(2-methylpropyl)oxy]phenyl}-methyl)-2-furanyl]-5-(1,1-dimethylethyl)-1H-benzimidazole | LC/MS Rt = 3.20min, [MH⁺] 437,439. |
| 128 | 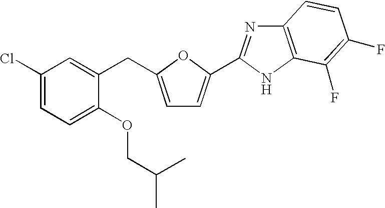 | 2-[5-({5-Chloro-2-[(2-methylpropyl)oxy]phenyl}-methyl)-2-furanyl]-4,5-difluoro-1H-benzimidazole | LC/MS Rt = 3.78min, [MH⁺] 417,419. |

### Example 129

### 2-[5-({5-Chloro-2-[(phenylmethyl)oxy]phenyl}methyl)-2-furanyl]-5,6-difluoro-1H-benzimidazole

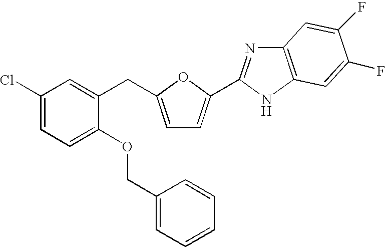

5-({5-Chloro-2-[(2-methylpropyl)oxy]phenyl}methyl)-2-furancarbaldehyde (65 mg, 0.2 mmol) and 4,5-difluoro-1,2-phenylenediamine (29 mg, 0.2 mmol) were dissolved in N,N-dimethylacetamide (2 ml) and the stirred mixture heated at 140° C. under argon for 2 h. Diethyl ether was added and the solution washed with water (×2), dried (MgSO₄) and evaporated. The residue was purified by flash chromatography, eluting with 5-30% ethyl acetate in hexane to give the title compound (22 mg).

LC/MS Rt=3.59 min, [MH⁺] 451, 453.

The following examples were prepared in a similar manner to 2-[5-({5-chloro-2-[(phenylmethyl)oxy]phenyl}methyl)-2-furanyl]-5,6-difluoro-1H-benzimidazole from the appropriate intermediates.

| Example | | Name | Data |
|---|---|---|---|
| 130 | 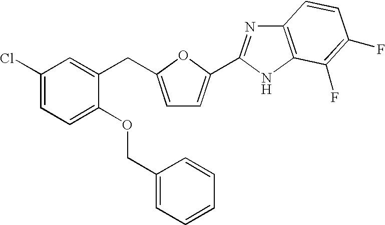 | 2-[5-({5-Chloro-2-[(phenylmethyl)oxy]-phenyl}methyl)-2-furanyl]-4,5-difluoro-1H-benzimidazole | LC/MS Rt =3.67 min,[MH⁺] 451,453. |
| 131 | 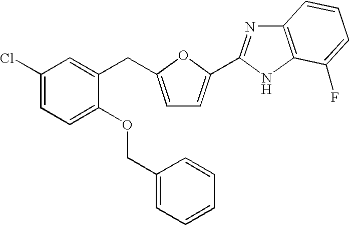 | 2-[5-({5-Chloro-2-[(phenylmethyl)oxy]-phenyl}methyl)-2-furanyl]-4-fluoro-1H-benzimidazole | LC/MS Rt =3.49 min,[MH⁺] 433,435. |

### Example 132

### {2-[5-({5-Chloro-2-[(2-methylpropyl)oxy]phenyl}methyl)-2-furanyl-1H-benzimidazol-5-yl}methanol

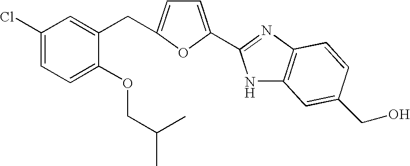

1M Lithium aluminium hydride in ether (2 ml) was added to a stirred solution of methyl 2-[5-({5-chloro-2-[(2-methylpropyl)oxy]phenyl}methyl)-2-furanyl]-1H-benzimidazole-5-carboxylate (500 mg, 1.14 mmol) in tetrahydrofuran (10 ml) under argon. After 2 hours the mixture was quenched with 2M sodium hydroxide and extracted with ethyl acetate. The organic phase was dried (magnesium sulphate), evaporated and purified by chromatography on silica gel eluting with ethyl acetate/hexane (2:1) to give the title compound as a white solid after trituration with ether (440 mg).

LC/MS: Rt=2.56, [MH]⁺ 411.3, 413.3

### Example 133

### {2-[5-({5-Chloro-2-[(phenylmethyl)oxy]phenyl}methyl)-2-furanyl]-1H-benzimidazol-5-yl}methanol

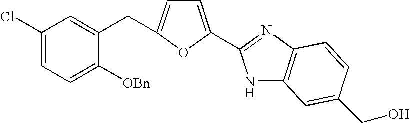

Prepared by the same method as for {2-[5-({5-chloro-2-[(2-methylpropyl)oxy]phenyl}methyl)-2-furanyl]-1H-benzimidazol-5-yl}methanol but using methyl 2-[5-({5-chloro-2-[(phenylmethyl)oxy]phenyl}methyl)-2-furanyl]-1H-benzimidazole-5-carboxylate instead of methyl 2-[5-({5-chloro-2-[(2-methylpropyl)oxy]phenyl}methyl)-2-furanyl]-1H-benzimidazole-5-carboxylate. LC/MS: Rt=2.53, [MH]⁺ 445.2, 447.2.

### Example 134

### {2-[5-({5-chloro-2-[(2-methylpropyl)oxy]phenyl}methyl)-2-furanyl]-1H-benzimidazol-4-yl}methanol

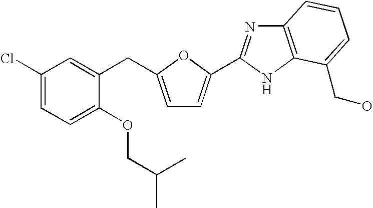

Prepared by the same method as for {2-[5-({5-chloro-2-[(2-methylpropyl)oxy]phenyl}-methyl)-2-furanyl]-1H-benzimidazol-5-yl}methanol but using methyl 2-[5-({5-chloro-2-[(2-methylpropyl)oxy]phenyl}methyl)-2-furanyl]-1H-benzimidazole-4-carboxylate instead of methyl 2-[5-({5-chloro-2-[(2-methylpropyl)oxy]phenyl}methyl)-2-furanyl]-1H-benzimidazole-5-carboxylate. LC/MS. Rt=2.64 min. [M+H] 411 1Cl.

### 2-[5-({5-Chloro-2-[(2-methylpropyl)oxy]phenyl}methyl)-2-furanyl]-1H-benzimidazole-5-carbaldehyde

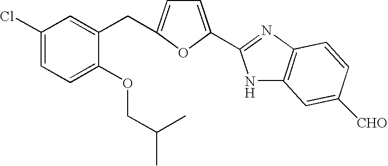

Dess-Martin periodinane (424 mg, 1 mmol) was added to a stirred solution of {2-[5-({5-chloro-2-[(2-methylpropyl)oxy]phenyl}methyl)-2-furanyl]-1H-benzimidazol-5-yl}methanol (390 mg, 0.95 mmol) in dichloromethane (10 ml) under argon. After 1 hour the solution was washed with saturated sodium bicarbonate, dried (magnesium sulphate), evaporated and purified by chromatography on silica gel eluting with ethyl acetate/hexane (1:2) to give the title compound as a white solid (266 mg). LC/MS: Rt=3.43, [MH]⁺ 409.2, 411.2.

### 2-[5-({5-Chloro-2-[(phenylmethyl)oxy]phenyl}methyl)-2-furanyl]-1H-benzimidazole-5-carbaldehyde

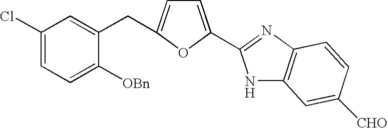

Dess-Martin periodinane (848 mg, 2 mmol) was added to a stirred solution of {2-[5-({5-chloro-2-[(phenylmethyl)oxy]phenyl}methyl)-2-furanyl]-1H-benzimidazol-5-yl}methanol (720 mg, 1.62 mmol) in dichloromethane (20 ml) under argon. After 1 hour the solution was washed with saturated sodium bicarbonate, dried (magnesium sulphate), evaporated and purified by chromatography on silica gel eluting with ethyl acetate/hexane (45:55) to give the title compound as a white solid after trituration with ether (516 mg).

LC/MS: Rt=3.36, [MH]⁺ 443.2, 445.2.

### {2-[5-({5-Chloro-2-[(2-methylpropyl)oxy]phenyl}methyl)-2-furanyl]-1H-benzimidazol-5-yl}acetaldehyde

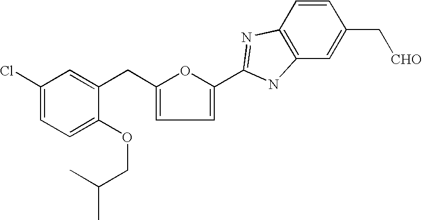

Dess-Martin periodinane (636 mg, 1.5 mmol) was added to a stirred solution of 2-{2-[5-({5-chloro-2-[(2-methylpropyl)oxy]phenyl}methyl)-2-furanyl]-1H-benzimidazol-5-yl}ethanol (425 mg, 1 mmol) in dichloromethane (10 ml) under argon. After 30 minutes the solution was washed with a solution of sodium thiosulphate in saturated sodium bicarbonate, dried (magnesium sulphate), evaporated and purified by chromatography on silica gel eluting with ethyl acetate/hexane (1:1) to give the title compound as a pale coloured gum (390 mg). LC/MS: Rt=2.90, [MH]⁺ 423.23, 425.23.

### Example 135

### 6-Bromo-2-[5-({5-chloro-2-[(2-methylpropyl)oxy]phenyl}methyl)-2-furanyl]-1H-imidazo[4,5-b]pyridine hydrochloride

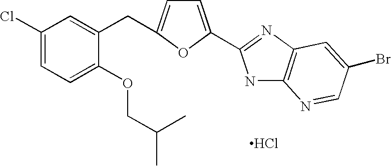

Oxalyl chloride (4 ml) was added to a solution of 5-({5-chloro-2-[(2-methylpropyl)oxy]phenyl}methyl)-2-furancarboxylic acid (3.5 g, 11.35 mmol) and one drop of DMF in dichloromethane (50 ml) and left at room temperature for one hour. The resulting solution was evaporated to dryness, azeotroped with toluene and the residue dissolved in dichloromethane (10 ml) and added to a stirred solution of 2,3-diamino-5-bromopyridine (4.27 g, 22.7 mmol) in 1:3:1 pyridine/dichloromethane/DMF (50 ml) and stirred for 30 minutes. The solution was diluted with ether/water and the organic phase washed with water, dried with magnesium sulphate, evaporated, azeotroped with toluene and chromatographed on silica gel eluting with 1:2 ethyl acetate/hexane. The product was triturated with 1:2 ether/hexane and filtered off to give 4.45 g of white solid which was dissolved in acetic acid (40 ml) and stirred and refluxed for 72 hours. The resulting solution was evaporated and water/ethyl acetate added. The solid was filtered off and dried to give the title compound a pink solid (2.1 g). 40 mg of the pink solid were dissolved in methanol/dichloromethane and hydrogen chloride in ether (1 ml) was added. The solvent was evaporated and the residue triturated with ether and filtered off to give the title compound as a white solid. LC/MS: Rt=3.72, [MH]⁺ 462.0, 464.0.

### Example 136

### 5-Bromo-2-[5-({5-chloro-2-[(2-methylpropyl)oxy]phenyl}methyl)-2-furanyl]-1H-imidazo[4,5-b]pyrazine hydrochloride

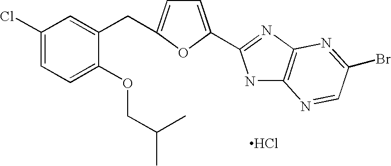

Oxalyl chloride (3 ml) was added to a solution of 5-({5-chloro-2-[(2-methylpropyl)oxy]-phenyl}methyl)-2-furancarboxylic acid (3.09 g, 10 mmol) and one drop of DMF in dichloromethane (30 ml) and left at room temperature for one hour. The resulting solution was evaporated to dryness, azeotroped with toluene and the residue dissolved in dichloromethane (15 ml) and added dropwise to a stirred solution of 2,3-diamino-5-bromopyrazine (2.08 g, 11 mmol) and 4-dimethyaminopyridine (100 mg, 0.82 mmol) in pyridine (20 ml) and stirred for one hour. The solution was evaporated and the residue dissolved in ether/water. The organic phase was dried with magnesium sulphate, evaporated and chromatographed on silica gel eluting with ethyl acetate/hexane (15:85) to give 3.35 g of white solid which was dissolved in acetic acid (40 ml) and stirred and refluxed for 5 days. The resulting solution was evaporated, dissolved in ethyl acetate and washed with 2M sodium hydroxide solution. The organic phase was dried with magnesium sulphate, evaporated and purified by flash chromatography on silica eluting with ethyl acetate/hexane (1:3) to give a pale yellow solid. A sample was dissolved in methanol/dichloromethane and hydrogen chloride in ether (1 ml) was added. The solvent was evaporated and the residue triturated with ether and filtered off to give the title compound as a solid. LC/MS: Rt=3.67, [MH]⁺ 463.11, 464.16.

### Example 137

### 2-[5-({5-Chloro-2-[(2-methylpropyl)oxy]phenyl}methyl)-2-furanyl]-6-ethenyl-1H-imidazo[4,5-b]pyridine hydrochloride

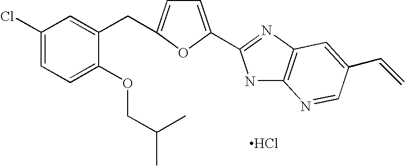

A suspension of 6-bromo-2-[5-({5-chloro-2-[(2-methylpropyl)oxy]phenyl}methyl)-2-furanyl]-1H-imidazo[4,5-b]pyridine hydrochloride (230 mg, 0.5 mmol) and tetrakis(triphenylphosphine)palladium(0) (58 mg, 0.05 mmol) in DME (5 ml) was stirred at room temperature under argon for 20 minutes then a solution vinylboronic anhydride pyridine complex (120 mg, 0.5 mmol) in DME (1 ml) and a solution of potassium carbonate (69 mg, 0.5 mmol) in water (0.75 ml) were added. The resulting mixture was stirred and refluxed for 24 hours then cooled, diluted with ethyl acetate/water and the organic phase dried with magnesium sulphate, evaporated and purified by flash chromatography on silica eluting with ethyl acetate/hexane (2:3). The product was dissolved in dichloromethane and hydrogen chloride in ether (1 ml) was added. The solvent was evaporated and the residue triturated with ether and filtered off to give the title compound as a pale yellow solid (30 mg). LC/MS: Rt=3.48, [MH]⁺ 408.29, 410.28.

### 2-[5-(555-Chloro-2-[(2-methylpropyl)oxy]phenyl}methyl)-2-furanyl]-1H-imidazo[4,5-b]pyridine-6-carbaldehyde

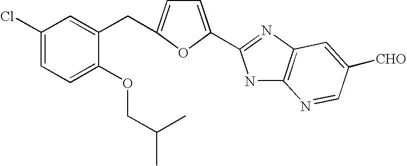

Sodium periodate (43 mg, 0.2 mmol) was added to a stirred mixture of 2-[5-({5-chloro-2-[(2-methylpropyl)oxy]phenyl}methyl)-2-furanyl]-6-ethenyl-1H-imidazo[4,5-b]pyridine (41 mg, 0.1 mmol) in acetonitrile (3 ml), water (0.5 ml) and 3.5% ruthenium (III) chloride aqueous solution (0.1 ml) and stirred for 20 hours). The mixture was diluted with water/ethyl acetate and the organic phase dried with magnesium sulphate, evaporated and purified by flash chromatography on silica eluting with ethyl acetate/hexane (2:3) to give the title compound as a white solid (16 mg). LC/MS: Rt=3.33, [MH]⁺ 410.21, 412.21.

### Example 138

### ({2-[5-({5-Chloro-2-[(phenylmethyl)oxy]phenyl}methyl)-2-furanyl]-1H-benzimidazol-5-yl}methyl)dimethylamine dihydrochloride

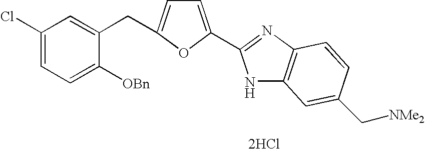

Sodium triacetoxyborohydride (85 mg, 0.4 mmol) was added to a solution of 2M dimethylamine in tetrahydrofuran (0.25 ml, 0.5 mmol) and 2-[5-({5-chloro-2-[(phenylmethyl)oxy]phenyl}methyl)-2-furanyl]-1H-benzimidazole-5-carbaldehyde (89 mg, 0.2 mmol) in tetrahydrofuran (3 ml). After 3 hours at room temperature the solution was diluted with ethyl acetate/water and the organic phase dried (magnesium sulphate), evaporated and purified by chromatography on silica gel eluting with methanol/dichloromethane (1:3). The product was dissolved in dichloromethane (5 ml), 1M hydrogen chloride in ether (2 ml) added and the solution evaporated to dryness. The residue was triturated with ether to give the title compound as a white solid (51 mg).

LC/MS: Rt=2.27, [MH]⁺ 472.2.

The following compounds were prepared by the same method as above for ({2-[5-({5-chloro-2-[(phenylmethyl)oxy]phenyl}methyl)-2-furanyl]-1H-benzimidazol-5-yl}methyl)dimethylamine dihydrochloride using the appropriate amine and either 2-[5-({5-chloro-2-[(phenylmethyl)oxy]phenyl}methyl)-2-furanyl]-1H-benzimidazole-5-carbaldehyde or 2-[5-({5-chloro-2-[(2-methylpropyl)oxy]phenyl}methyl)-2-furanyl]-1H-benzimidazole-5-carbaldehyde or {2-[5-({5-chloro-2-[(2-methylpropyl)oxy]phenyl}methyl)-2-furanyl]-1H-benzimidazol-5-yl}acetaldehyde or 2-[5-({5-chloro-2-[(2-methylpropyl)oxy]phenyl}methyl)-2-furanyl]-1H-imidazo[4,5-b]pyridine-6-carbaldehyde.

| Example | | Name | Data |
|---|---|---|---|
| 139 | 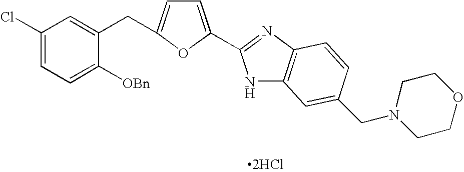 | 2-[5-({5-Chloro-2-[(phenylmethyl)oxy]-phenyl}methyl)-2-furanyl]-5-(4-morpholinylmethyl)-1H-benzimidazoledihydrochloride | LC/MSRt = 2.29[MH]⁺ 514.3 |
| 140 | 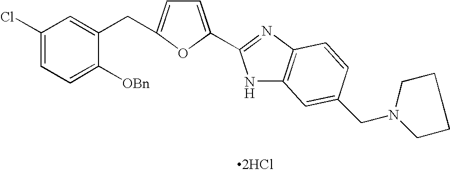 | 2-[5-({5-Chloro-2-[(phenylmethyl)oxy]-phenyl}methyl)-2-furanyl]-5-(1-pyrrolidinylmethyl)-1H-benzimidazoledihydrochloride | LC/MSRt = 2.33[MH]⁺ 498.3 |
| 141 | 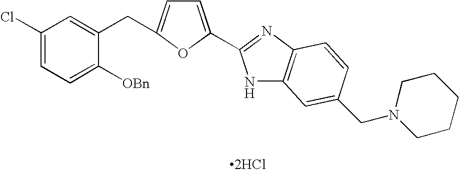 | 2-[5-({5-Chloro-2-[(phenylmethyl)oxy]-phenyl}methyl)-2-furanyl]-5-(1-piperidinylmethyl)-1H-benzimidazoledihydrochloride | LC/MSRt = 2.38[MH]⁺ 512.3 |
| 142 | 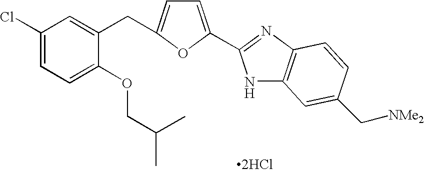 | ({2-[5-({5-Chloro-2-[(2-methylpropyl)oxy]-phenyl}methyl)-2-furanyl]-1H-benzimidazol-5-yl}methyl)dimethyl-amine dihydrochloride | LC/MSRt = 2.30[MH]⁺ 438.3 |
| 143 | 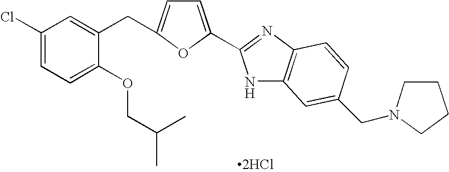 | 2-[5-({5-Chloro-2-[(2-methylpropyl)oxy]-phenyl}methyl)-2-furanyl]-5-(1-pyrrolidinylmethyl)-1H-benzimidazoledihydrochloride | LC/MSRt = 2.31[MH]⁺ 464.3,466.2 |
| 144 | 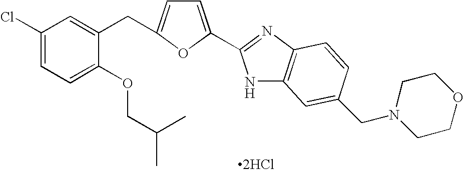 | 2-[5-({5-Chloro-2-[(2-methylpropyl)oxy]-phenyl}methyl)-2-furanyl]-5-(4-morpholinylmethyl)-1H-benzimidazoledihydrochloride | LC/MSRt = 2.31[MH]⁺ 480.3 |
| 145 | 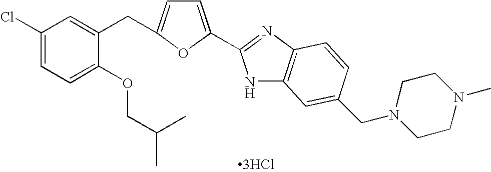 | 2-[5-({5-Chloro-2-[(2-methylpropyl)oxy]-phenyl}methyl)-2-furanyl]-5-[(4-methyl-1-piperazinyl)methyl]-1H-benzimidazoletrihydrochloride | LC/MSRt = 2.17[MH]⁺ 493.3 |
| 146 | 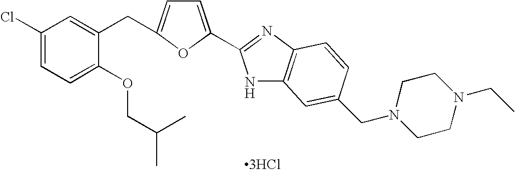 | 2-[5-({5-Chloro-2-[(2-methylpropyl)oxy]-phenyl}methyl)-2-furanyl]-5-[(4-ethyl-1-piperazinyl)methyl]-1H-benzimidazoletrihydrochloride | LC/MSRt = 2.18[MH]⁺ 507.3 |
| 147 | 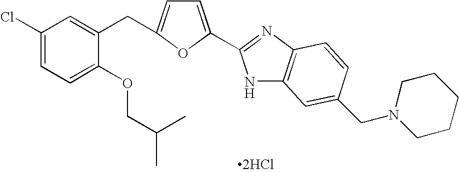 | 2-[5-({5-Chloro-2-[(2-methylpropyl)oxy]-phenyl}methyl)-2-furanyl]-5-(1-piperidinylmethyl)-1H-benzimidazoledihydrochloride | LC/MSRt = 2.43[MH]⁺ 478.3 |
| 148 | 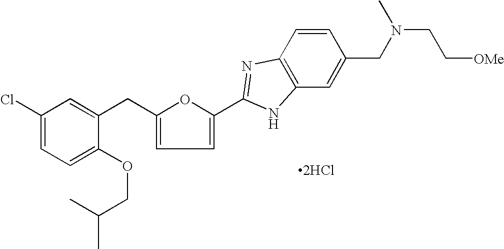 | N-({2-[5-({5-Chloro-2-[(2-methylpropyl)oxy]-phenyl}methyl)-2-furanyl]-1H-benzimidazol-5-yl}methyl)-N-methyl-2-(methyloxy)ethanaminedihydrochloride | LC/MSRt = 2.57[MH]⁺ 424.3,426.3 |
| 149 | 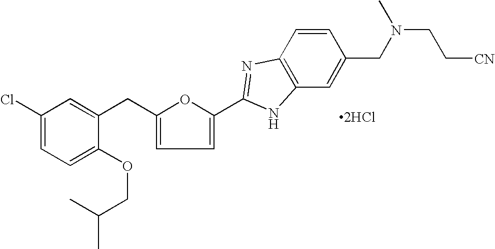 | 3-[({2-[5-({5-Chloro-2-[(2-methylpropyl)oxy]-phenyl}methyl)-2-furanyl]-1H-benzimidazol-5-yl}methyl)(methyl)-amino]propanenitriledihydrochloride | LC/MSRt = 2.40[MH]⁺ 477.3 |
| 150 | 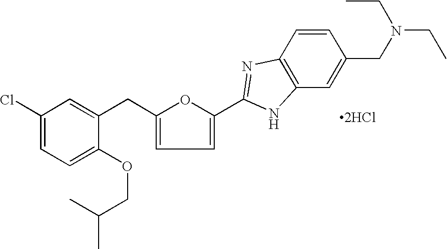 | N-({2-[5-({5-Chloro-2-[(2-methylpropyl)oxy]-phenyl}methyl)-2-furanyl]-1H-benzimidazol-5-yl}methyl)-N-ethylethanaminedihydrochloride | LC/MSRt = 2.43[MH]⁻ 464.18,466.18 |
| 151 | 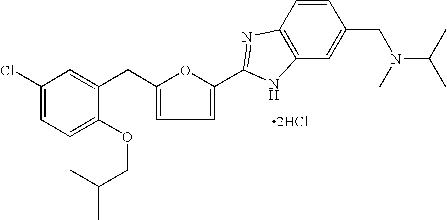 | N-({2-[5-({5-Chloro-2-[(2-methylpropyl)oxy]-phenyl}methyl)-2-furanyl]-1H-benzimidazol-5-yl}methyl)-N-methyl-2-propanaminedihydrochloride | LC/MSRt = 2.42[MH]⁻ 464.19,466.19 |
| 152 | 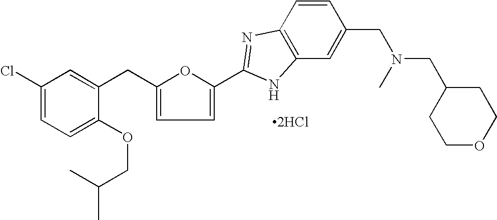 | ({2-[5-({5-Chloro-2-[(2-methylpropyl)oxy]-phenyl}methyl)-2-furanyl]-1H-benzimidazol-5-yl}methyl)methyl(tetrahydro-2H-pyran-4-ylmethyl)aminedihydrochloride | LC/MSRt = 2.42[MH]⁻ 520.24,522.20 |
| 153 | 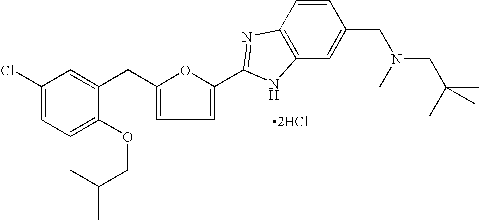 | N-({2-[5-({5-Chloro-2-[(2-methylpropyl)oxy]-phenyl}methyl)-2-furanyl]-1H-benzimidazol-5-yl}methyl)-N,2,2-trimethyl-1-propanaminedihydrochloride | LC/MSRt = 2.57[MH]⁻ 492.24,494.25 |
| 154 | 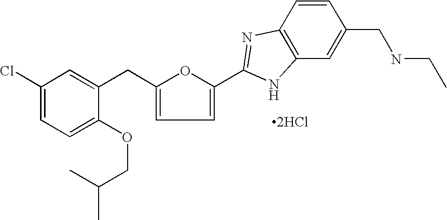 | N-({2-[5-({5-Chloro-2-[(2-methylpropyl)oxy]-phenyl}methyl)-2-furanyl]-1H-benzimidazol-5-yl}methyl)ethanaminedihydrochloride | LC/MSRt = 2.29[MH]⁻ 436.17,438.16 |
| 155 | 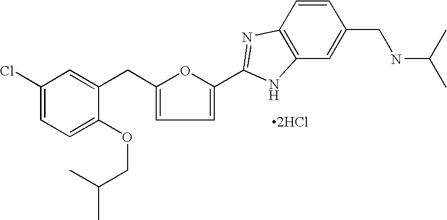 | N-({2-[5-({5-Chloro-2-[(2-methylpropyl)oxy]-phenyl}methyl)-2-furanyl]-1H-benzimidazol-5-yl}methyl)-2-propanaminedihydrochloride | LC/MSRt = 2.33[MH]⁻ 450.15,452.18 |
| 156 | 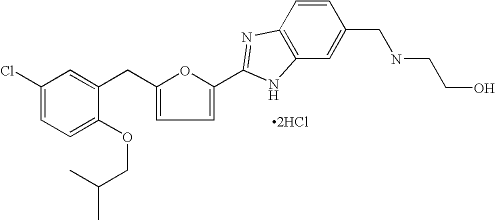 | 2-[({2-[5-({5-Chloro-2-[(2-methylpropyl)oxy]-phenyl}methyl)-2-furanyl]-1H-benzimidazol-5-yl}methyl)amino]-ethanol | LC/MSRt = 2.24[MH]⁻ 452.16,454.14 |
| 157 | 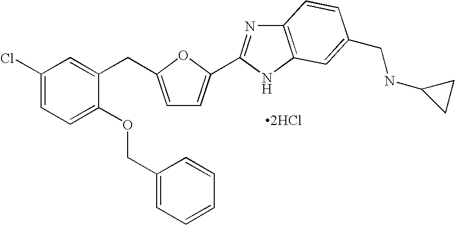 | N-({2-[5-({5-Chloro-2-[(phenylmethyl)oxy]-phenyl}methyl)-2-furanyl]-1H-benzimidazol-5-yl}methyl)cyclopropan-aminedihdrochloride | LC/MS. Rt =2.28 min.[M + H] 484 1 Cl |
| 158 | 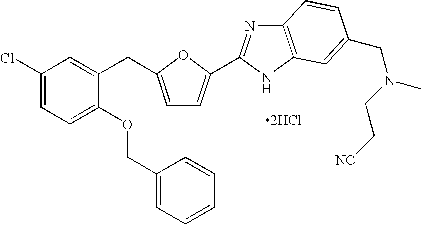 | 3-[({2-[5-({5-Chloro-2-[(phenylmethyl)oxy]-phenyl}methyl)-2-furanyl]-1H-benzimidazol-5-yl}methyl)(methyl)-amino]propanenitriledihydrochloride | LC/MS. Rt =2.35 min.[M + H] 511 1 Cl |
| 159 | 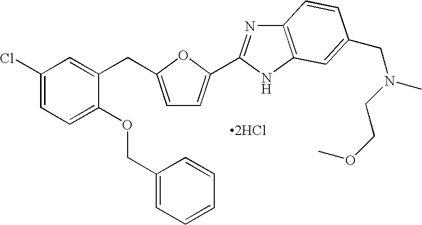 | N-({2-[5-({5-Chloro-2-[(phenylmethyl)oxy]-phenyl}methyl)-2-furanyl]-1H-benzimidazol-5-yl}methyl)-N-methyl-2-(methyloxy)ethan-amine dihydrochloride | LC/MS. Rt =2.35 min.[M + H] 516 1 Cl |
| 160 | 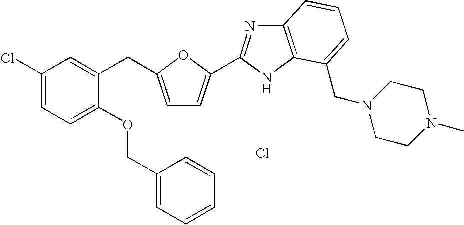 | 2-[5-({5-Chloro-2-[(phenylmethyl)oxy]-phenyl}methyl)-2-furanyl]-4-[(4-methyl-1-piperazinyl)methyl]-1H-benzimidazolehydrochloride | LC/MS. Rt =2.11 min.[M + H] 527 1 Cl |
| 161 | 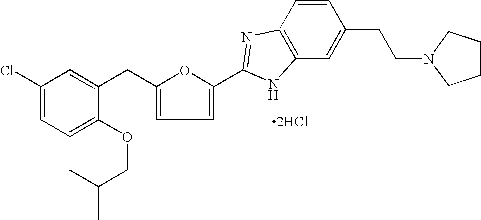 | 2-[5-({5-Chloro-2-[(2-methylpropyl)oxy]-phenyl}methyl)-2-furanyl]-5-[2-(1-pyrrolidinyl)ethyl]-1H-benzimidazoledihydrochloride | LC/MSRt = 2.31[MH]⁻ 476.19,478.11 |
| 162 | 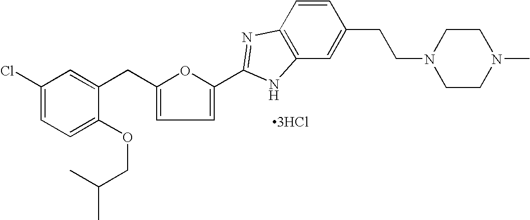 | 2-[5-({5-Chloro-2-[(2-methylpropyl)oxy]-phenyl}methyl)-2-furanyl]-5-[2-(4-methyl-1-piperazinyl)ethyl]-1H-benzimidazoletrihydrochloride | LC/MSRt = 2.09[MH]⁻ 505.22,507.21 |
| 163 | 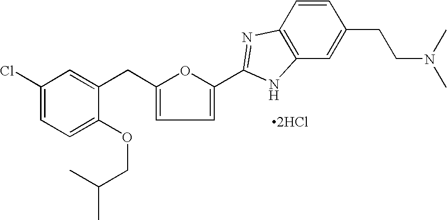 | (2-{2-[5-({5-Chloro-2-[(2-methylpropyl)oxy]-phenyl}methyl)-2-furanyl]-1H-benzimidazol-5-yl}ethyl)dimethylaminedihydrochloride | LC/MSRt = 2.17[MH]⁻ 450.14,452.15 |
| 164 | 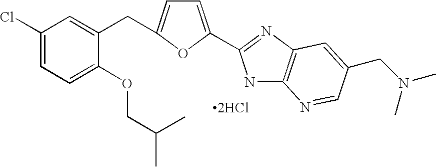 | ({2-[5-({5-Chloro-2-[(2-methylpropyl)oxy]-phenyl}methyl)-2-furanyl]-1H-imidazo[4,5-b]pyridin-6-yl}methyl)dimethyl-amine dihydrochloride | LC/MSRt = 2.27[MH]⁺ 439.25,441.24 |
| 165 | 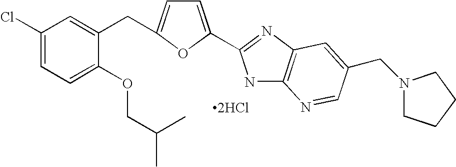 | 2-[5-({5-Chloro-2-[(2-methylpropyl)oxy]-phenyl}methyl)-2-furanyl]-6-(1-pyrrolidinylmethyl)-1H-imidazo[4,5-b]pyridinedihydrochloride | LC/MSRt = 2.34[MH]⁺ 465.2,467.1 |

### Example 166

### ({2-[5-({5-Chloro-2-[(2-methylpropyl)oxy]phenyl}methyl)-2-furanyl]-1H-benzimidazol-5-yl}methyl)methylamine dihydrochloride

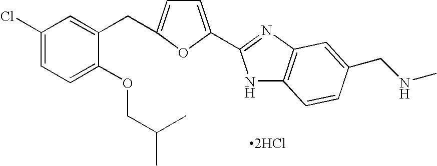

Sodium triacetoxyborohydride (85 mg, 0.4 mmol) was added to a 33% solution of methylamine in ethanol (0.1 ml, 1 mmol) and 2-[5-({5-chloro-2-[(2-methylpropyl)oxy]phenyl}methyl)-2-furanyl]-1H-benzimidazole-5-carbaldehyde (82 mg, 0.2 mmol) in tetrahydrofuran (3 ml). After 2 hours at room temperature sodium borohydride (19 mg, 0.5 mmol) was added and the mixture stirred for 1 hour. The resulting solution was diluted with ethyl acetate/water and the organic phase dried (magnesium sulphate), evaporated and purified by flash chromatography on silica gel eluting with methanol/dichloromethane (1:19) to remove impurities then with methanol/dichloromethane (1:1). The product was dissolved in dichloromethane (5 ml), 1M hydrogen chloride in ether (2 ml) added and the solution evaporated to dryness. The residue was triturated with ether to give the title compound as a white solid (39 mg).

LC/MS: Rt=2.27, [MH]⁺ 424.3

### Example 167

### 2-[5-({5-Chloro-2-[(2-methylpropyl)oxy]phenyl}methyl)-2-furanyl]-1H-imidazole

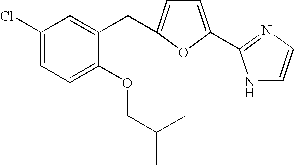

A mixture of aminoacetaldehyde dimethyl acetal (93 mg, 0.89 mmol) and methyl 5-({5-chloro-2-[(2-methylpropyl)oxy]phenyl}methyl)-2-furancarboximidoate_hydrochloride (248 mg, 0.67 mmol) in ethanol (3 ml) was stirred and refluxed for 5 hours then cooled and evaporated to give a colourless gum which was dissolved in 1:1 THF/2M hydrochloric acid (5 ml) and heated at 60° C. for 5 hours. The resulting solution was cooled, diluted with ether/2M sodium hydroxide and the organic phase dried (magnesium sulphate), evaporated and purified by MDAP. The product was dissolved in ether/2M sodium hydroxide and the organic phase dried (magnesium sulphate), evaporated and triturated with ether/hexane to give the title compound as a white solid (73 mg).

LC/MS: Rt=2.25, [MH]⁺ 331.4, 333.5

### Example 168

### 3-[5-({5-Chloro-2-[(2-methylpropyl)oxy]phenyl}methyl)-2-furanyl-1H-pyrazole

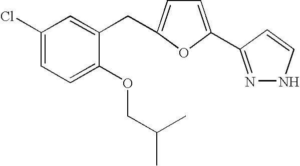

A mixture of dimethylformamide dimethyl acetal (412 mg, 3.46 mmol) and 1-[5-({5-chloro-2-[(2-methylpropyl)oxy]phenyl}methyl)-2-furanyl]ethanone (590 mg, 1.73 mmol) was stirred and heated at 100° C. under argon for 16 hours then dissolved in toluene (2 ml), evaporated to dryness and the residue dissolved in methanol (2 ml) and hydrazine hydrate (0.5 ml). After refluxing for 1 hour the solution was diluted with water/diethyl ether and the organic phase dried (magnesium sulphate), evaporated and purified by chromatography on silica gel eluting with ethyl acetate/hexane (1:2) to give the title compound as a light brown solid after trituration with ether (241 mg). LC/MS: Rt=3.40, [MH]⁺ 365.0, 367.0.

### 5-({5-Chloro-2-[(phenylmethyl)oxy]phenyl}methyl)-N-[(1E)-(dimethylamino)methylidene]-2-furancarboxamide

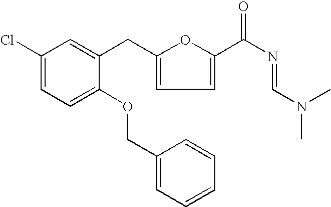

A solution of 5-({5-chloro-2-[(phenylmethyl)oxy]phenyl}methyl)-2-furancarboxamide (200 mg, 0.88 mmol) in dimethylformamide dimethyl acetal (2 ml) was heated at 120° C. for 1 hour. The product crystallised on standing. The title compound was collected by filtration as a colourless solid (300 mg). LC/MS: Rt=2.97 min, [MH]⁺ 397.

### Example 169

### 3-[5-({5-Chloro-2-[(phenylmethyl)oxy]phenyl}methyl)-2-furanyl]-1H-1,2,4-triazole

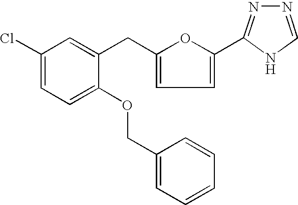

A mixture of 5-({5-chloro-2-[(phenylmethyl)oxy]phenyl}methyl)-N-[(1E)-(dimethylamino)methylidene]-2-furancarboxamide (200 mg, 0.5 mmol) and hydrazine hydrate (30 mg, 0.6 mmol) in glacial acetic acid (1 ml) and 1,4-dioxan (1 ml) was heated at 120° C. for 1 hour. The mixture was cooled and the solvent evaporated. The residue was purified by reverse phase chromatography eluting with 5-100% acetonitrile/water to give the title compound as a colourless solid (70 mg). LC/MS: Rt=3.02 min, [MH]⁺ 366.

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at Ep₁ and Ep₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1(CHO-K1) cells into which a stable (pCIN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20 mins and the pellet containing the membrane fraction is washed (optional) three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

### Biological Activity at TP Receptor

To determine if a compound has agonist or antagonist activity at the TP receptor a functional calcium mobilisation assay may be performed. Briefly, the antagonist properties of compounds are assessed by their ability to inhibit the mobilisation of intracellular calcium ([Ca²⁺]ᵢ) in response to activation of TP receptors by the stable TXA₂ mimetic U46619 (9,11-dideoxy-11α, 9α-epoxy-methanoprostaglandin F2 α; commercially available from e.g. Sigma-Aldrich). Increasing concentrations of antagonist reduce the amount of calcium that a given concentration of U46619 can mobilise. The net effect is to displace the U46619 concentration-effect curve. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software. The agonist activity of the compounds are determined by their ability to cause an increase in intracellular mobilisation in the absence of U46619.

The human TP calcium mobilisation assay utilises Chinese hamster ovary-K1 (CHO-K1) cells into which a stable (pCIN; BioTechniques 20 (1996): 102-110) vector containing TP cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 96-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of U46619 are then added to the plate in order to assess the antagonist properties of the compounds.

The data so generated may be analysed by means of a computerised curve-fitting routine. The concentration of compound that elicits a half-maximal inhibition of the calcium mobilisation induced by U46619 (pIC₅₀) may then be estimated, and the percentage activation caused by the compounds directly can be used to determine if there is any agonism present.

### Results

The compounds of examples 1-169 were tested in the binding assay for the human prostanoid EP₁ receptor. The results are expressed as pIC₅₀ values. A pIC₅₀ is the negative logarithm₁₀ of the IC₅₀. The results given are averages of a number of experiments. The compounds of the examples had a pIC₅₀ value ≧6. More particularly, the compounds of the examples 3, 5-7, 10,15,18-23, 25, 35, 36, 57, 84, 98, 99, 104-108, 111-113, 115, 117, 132, 133, 136, 142, 143, 145-149, 151, 156, 162, and 166 exhibited a pIC₅₀ value ≧8.

The compounds of examples 1-58, 67-129, 131-156 and 160-169 (free bases or sodium salts) were tested in the human EP₁ calcium mobilisation assay. The results are expressed as functional pKᵢ values. A functional pKi is the negative logarithm₁₀ of the antagonist dissociation constant as determined in the human EP₁ calcium mobilisation assay. The results given are averages of a number of experiments. The compounds of examples 1-12, 14-74, 76-109, 111-133 and 137-169 exhibited a functional pKi value >6. More particularly, the compounds of examples 5, 7, 14-18, 21-26, 28, 35, 36, 53, 57, 79, 98, 104, 106, and 167 exhibited a functional pKi value of ≧8. The compounds of examples 13, 75, 110, and 134-136 exhibited a functional pKi value <6.

The compounds of examples 1-58, 67-98, 100-129, 131-156 and 160-169 (free bases or sodium salts) were tested in the human EP₃ calcium mobilisation assay. The results are expressed as functional pKᵢ values. A functional pKi is the negative logarithms of the antagonist dissociation constant as determined in the human EP₃ calcium mobilisation assay. The results given are averages of a number of experiments. The compounds of examples 1-5, 7-58, 67-90, 93-97, 101, 103-107, 110-118, 11-129, 131-156 and 160-169 exhibited a functional pKi value of ≦6.5. The compounds of examples 2, 3, 8-10, 17, 19, 20, 26, 27, 33, 34, 45, 55, 69, 70, 72-74, 76, 80, 88, 89, 96, 110, 111, 114, 116, 126-128, 134, 135, 137-140, 143,145-147, 150-154, 156 and 160-166 showed no activity in a functional assay.

No toxicological effects were observed in these tests.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 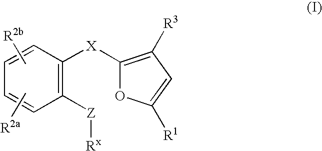
wherein:
X is CR⁷R⁸, O, S, SO, or SO₂;
Z is O, S, SO or SO₂;
R^{x} is C₂₋₁₀alkyl optionally substituted by C₁₋₃alkoxy, optionally substituted C₂₋₁₀alkenyl, optionally substituted C₂₋₁₀alkynyl, optionally substituted CQ^{a}Q^{b}-heterocyclyl, optionally substituted CQ^{a}Q^{b}-bicyclic heterocyclyl, or optionally substituted CQ^{a}Q^{b}-aryl;
R¹ is CO₂H, CONR⁴R⁵, CH₂CO₂H, NHCO₂R⁶, 1,2,4-triazolyl, tetrazolyl, or CH₂tetrazolyl; or R¹ is imidazolyl, or pyrazolyl wherein optionally the imidazole or pyrazole ring is fused to give an optionally substituted bicyclic or tricyclic ring system;
R^{2a} and R^{2b} are independently selected from hydrogen, halo, CN, SO₂alkyl, SR⁴, NO₂, optionally substituted alkyl, optionally substituted alkoxy, optionally substituted aryl, and optionally substituted heteroaryl;
R³ is hydrogen or optionally substituted C₁₋₃alkyl;
R⁴ is hydrogen or optionally substituted alkyl;
R⁵ is hydrogen or optionally substituted alkyl, optionally substituted aryl, optionally substituted heteroaryl, optionally substituted SO₂aryl, optionally substituted SO₂alkyl, optionally substituted SO₂heteroaryl, optionally substituted CQ^{a}Q^{b} aryl, or optionally substituted CQ^{a}Q^{b} heteroaryl; or
R⁴ and R⁵ together with the nitrogen to which they are attached form a benzimidazolyl or 4-phenylmethylpiperazinyl group;
R⁶ is optionally substituted alkyl or optionally substituted aryl;
R⁷ is hydrogen, fluorine or alkyl;
R⁸ is hydrogen, hydroxy, fluorine or alkyl;
or R⁷ and R⁸ together with the carbon to which they are attached form a cycloalkyl ring, optionally containing up to one heteroatom selected from O, S, NH and N-alkyl; or R⁷ and R⁸ together with the carbon to which they are attached form a carbonyl group; and
Q^{a} and Q^{b} are each independently selected from hydrogen, CH₃ and fluorine;
Or a derivative thereof;
provided that:
when R¹ is NHCO₂R⁶, R^{x} represents optionally substituted alkyl;
when R¹ is benzimidazolyl it is unsubstituted on the 1-position; and
when R¹ is benzimidazolyl, optional substituents on the 4 or 7 position are selected from halogen, CH₂OH and CO₂H.

2. A compound according to claim 1 wherein X is CR⁷R⁸ or O.

3. A compound according to claim 1 wherein Z is O.

4. A compound according to claim 1 wherein R^{2a} is hydrogen, and R^{2b} is Cl and is positioned 1,4-relative to the Z substituent and 1,3-relative to the methylene furyl moiety.

5. (canceled)

6. A pharmaceutical composition comprising a compound according to claim 1 or a pharmaceutically acceptable derivative thereof together with a pharmaceutical carrier and/or excipient.

7. A compound according to claim 1 or a pharmaceutically acceptable derivative thereof for use as an active therapeutic substance.

8. (canceled)

9. (canceled)

10. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

11. A method of treating a human or animal subject suffering from inflammatory pain, neuropathic pain or visceral pain which method comprises administering to said subject an effective amount of a compound according to claim 1 or a pharmaceutically acceptable derivative thereof.

12. (canceled)

13. (canceled)

14. (canceled)
